(19) United States
(12) Patent Application Publication
(10) Pub. No.: US 2007/0292346 A1
(43) Pub. Date: Dec. 20, 2007

(54) Lng105 Antibody Composition and Methods of Use, and Use of Lng105 to Assess Lung Cancer Risk

(76) Inventors: Rong Fan, Redwood City, CA (US); Nam Kim, Santa Clara, CA (US); Robert L. Wolfert, Palo Alto, CA (US); Glenn Pilkington, Victoria (AU)

Correspondence Address: LICATA & TYRRELL P.C., 66 E. MAIN STREET, MARLTON, NJ, 08053, US

(21) Appl. No.: 10/593,799

(22) PCT Filed: Mar. 25, 2005
(86) PCT No.: PCT/US05/10085

§ 371 Date: Aug. 6, 2007

Related U.S. Application Data
(60) Provisional application No. 60/556,466, filed on Mar. 25, 2004.

(51) Int. Cl. A61K 51/10; A61K 39/395; A61P 35/00; C07K 16/30; C12N 5/22; G01N 33/566; G01N 33/574; C12P 21/04; C12N 5/12; C07K 16/18; A61K 49/00

(52) U.S. Cl. 424/1.49; 424/155.1; 424/9.1; 435/325; 435/7.23; 435/70.21; 436/501; 530/387.3; 530/388.8

(57) ABSTRACT

This invention relates to a method for assessing risk of lung and/or breast cancer. Specifically, in one embodiment it relates to utilizing Lng105 to determine the risk of lung cancer. Specific antibodies are disclosed.

This patent application claims the benefit of priority from U.S. Provisional Application Ser. No. 60/556,466, filed Mar. 25, 2004, the teachings of which are herein incorporated by reference in their entirety.

### FIELD OF THE INVENTION

The present invention relates to anti-Lng105 antibody compositions and methods of killing and/or detecting Lng105-expressing cells.

### BACKGROUND OF THE INVENTION

Throughout the last hundred years, the incidence of lung cancer has steadily increased, so much so that now in many countries, it is the most common cancer. In fact, lung cancer is the second most prevalent type of cancer for both men and women in the United States and is the most common cause of cancer death in both sexes. Lung cancer deaths have increased ten-fold in both men and women since 1930, primarily due to an increase in cigarette smoking, but also due to an increased exposure to arsenic, asbestos, chromates, chloromethyl ethers, nickel, polycyclic aromatic hydrocarbons and other agents. See Scott, Lung Cancer: A Guide to Diagnosis and Treatment, Addicus Books (2000) and Alberg et al., in Kane et al. (eds.) Biology of Lung Cancer, pp. 11-52, Marcel Dekker, Inc. (1998). The American Cancer Society estimates there will be over 172,570 new cases of lung and bronchus cancer in 2005. Additionally, there will be an estimated 163,510 deaths from lung and bronchus cancer in 2005. ACS Website: cancer with the extension.org of the world wide web.

Lung cancer may result from a primary tumor originating in the lung or a secondary tumor which has spread from another organ such as the bowel or breast. Although there are over a dozen types of lung cancer, over 90% fall into two categories: small cell lung cancer (SCLC) and non-small cell lung cancer (NSCLC). See Scott, supra. About 20-25% of all lung cancers are characterized as SCLC, while 70-80% are diagnosed as NSCLC. Id. A rare type of lung cancer is mesothelioma, which is generally caused by exposure to asbestos, and which affects the pleura of the lung. Lung cancer is usually diagnosed or screened for by chest x-ray, CAT scans, PET scans, or by sputum cytology. A diagnosis of lung cancer is usually confirmed by biopsy of the tissue. Id.

SCLC tumors are highly metastatic and grow quickly. By the time a patient has been diagnosed with SCLC, the cancer has usually already spread to other parts of the body, including lymph nodes, adrenals, liver, bone, brain and bone marrow. See Scott, supra; Van Houtte et al. (eds.), Progress and Perspective in the Treatment of Lung Cancer, Springer-Verlag (1999). Because the disease has usually spread to such an extent that surgery is not an option, the current treatment of choice is chemotherapy plus chest irradiation. See Van Houtte, supra. The stage of disease is a principal predictor of long-term survival. Less than 5% of patients with extensive disease that has spread beyond one lung and surrounding lymph nodes, live longer than two years. Id. However, the probability of five-year survival is three to four times higher if the disease is diagnosed and treated when it is still in a limited stage, i.e., not having spread beyond one lung. Id.

NSCLC is generally divided into three types: squamous cell carcinoma, adenocarcinoma and large cell carcinoma. Both squamous cell cancer and adenocarcinoma develop from the cells that line the airways; however, adenocarcinoma develops from the goblet cells that produce mucus. Large cell lung cancer has been thus named because the cells look large and rounded when viewed microscopically, and generally are considered relatively undifferentiated. See Yesner, Atlas of Lung Cancer, Lippincott-Raven (1998).

Secondary lung cancer is a cancer initiated elsewhere in the body that has spread to the lungs. Cancers that metastasize to the lung include, but are not limited to, breast cancer, melanoma, colon cancer and Hodgkin's lymphoma. Treatment for secondary lung cancer may depend upon the source of the original cancer. In other words, a lung cancer that originated from breast cancer may be more responsive to breast cancer treatments and a lung cancer that originated from the colon cancer may be more responsive to colon cancer treatments.

The stage of a cancer indicates how far it has spread and is an important indicator of the prognosis. In addition, staging is important because treatment is often decided according to the stage of a cancer. SCLC is divided into two stages: limited disease, i.e., cancer that can only be seen in one lung and in nearby lymph nodes; and extensive disease, i.e., cancer that has spread outside the lung to the chest or to other parts of the body. For most patients with SCLC, the disease has already progressed to lymph nodes or elsewhere in the body at the time of diagnosis. See Scott, supra. Even if spreading is not apparent on the scans, it is likely that some cancer cells may have spread away and traveled through the bloodstream or lymph system. In general, chemotherapy with or without radiotherapy is often the preferred treatment. The initial scans and tests done at first will be used later to see how well a patient is responding to treatment.

In contrast, non-small cell cancer may be divided into four stages. Stage I is highly localized cancer with no cancer in the lymph nodes. Stage II cancer has spread to the lymph nodes at the top of the affected lung. Stage III cancer has spread near to where the cancer started. This can be to the chest wall, the covering of the lung (pleura), the middle of the chest (mediastinum) or other lymph nodes. Stage IV cancer has spread to another part of the body. Stage I-III cancer is usually treated with surgery, with or without chemotherapy. Stage IV cancer is usually treated with chemotherapy and/or palliative care.

A number of chromosomal and genetic abnormalities have been observed in lung cancer. In NSCLC, chromosomal aberrations have been described on 3p, 9p, 11p, 15p and 17p, and chromosomal deletions have been seen on chromosomes 7, 11, 13 and 19. See Skarin (ed.), Multimodality Treatment of Lung Cancer, Marcel Dekker, Inc. (2000); Gemmill et al., pp. 465-502, in Kane, supra; Bailey-Wilson et al., pp. 53-98, in Kane, supra. Chromosomal abnormalities have been described on 1p, 3p, 5q, 6q, 8q, 13q and 17p in SCLC. Id. In addition, the loss of the short arm of chromosome 3p has also been seen in greater than 90% of SCLC tumors and approximately 50% of NSCLC tumors. Id.

A number of oncogenes and tumor suppressor genes have been implicated in lung cancer. See Mabry, pp. 391-412, in Kane, supra and Sclafani et al., pp. 295-316, in Kane, supra. In both SCLC and NSCLC, the p53 tumor suppressor gene is mutated in over 50% of lung cancers. See Yesner, supra. Another tumor suppressor gene, FHIT, which is found on chromosome 3p, is mutated by tobacco smoke. Id.; Skarin, supra. In addition, more than 95% of SCLCs and approximately 20-60% of NSCLCs have an absent or abnormal retinoblastoma (Rb) protein, another tumor suppressor gene. The ras oncogene (particularly K-ras) is mutated in 20-30% of NSCLC specimens and the c-erbB2 oncogene is expressed in 18% of stage 2 NSCLC and 60% of stage 4 NSCLC specimens. See Van Houtte, supra. Other tumor suppressor genes that are found in a region of chromosome 9, specifically in the region of 9p21, are deleted in many cancer cells, including p16^{INK4A} and p15^{INK4B}. See Bailey-Wilson, supra; Sclafani et al., supra. These tumor suppressor genes may also be implicated in lung cancer pathogenesis.

In addition, many lung cancer cells produce growth factors that may act in an autocrine or paracrine fashion on lung cancer cells. See Siegfried et al., pp. 317-336, in Kane, supra; Moody, pp. 337-370, in Kane, supra and Heasley et al, 371-390, in Kane, supra. In SCLC, many tumor cells produce gastrin-releasing peptide (GRP), which is a proliferative growth factor for these cells. See Skarin, supra. Many NSCLC tumors express epidermal growth factor (EGF) receptors, allowing NSCLC cells to proliferate in response to EGF. Insulin-like growth factor (IGF-I) is elevated in greater than 95% of SCLC and greater than 80% of NSCLC tumors; it is thought to function as an autocrine growth factor. Id. Finally, stem cell factor (SCF, also known as steel factor or kit ligand) and c-Kit (a proto-oncoprotein tyrosine kinase receptor for SCF) are both expressed at high levels in SCLC, and thus may form an autocrine loop that increases proliferation. Id.

Although the majority of lung cancer cases are attributable to cigarette smoking, most smokers do not develop lung cancer. Epidemiological evidence has suggested that susceptibility to lung cancer may be inherited in a Mendelian fashion, and thus have an inherited genetic component. Bailey-Wilson, supra. Thus, it is thought that certain allelic variants at some genetic loci may affect susceptibility to lung cancer. Id. One way to identify which allelic variants are likely to be involved in lung cancer susceptibility, as well as susceptibility to other diseases, is to look at allelic variants of genes that are highly expressed in lung.

The lung is susceptible to a number of other debilitating diseases as well, including, without limitation, emphysema, pneumonia, cystic fibrosis and asthma. See Stockley (ed.), Molecular Biology of the Lung, Volume I: Emphysema and Infection, Birkhauser Verlag (1999), hereafter Stockley I, and Stockley (ed.), Molecular Biology of the Lung, Volume II: Asthma and Cancer, Birkhauser Verlag (1999), hereafter Stockley II. The cause of many these disorders is still not well understood and there are few, if any, good treatment options for many of these noncancerous lung disorders. Thus, there remains a need to understand various noncancerous lung disorders and to identify treatments for these diseases.

The development and differentiation of lung tissue during embryonic development is also very important. All of the epithelial cells of the respiratory tract, including those of the lung and bronchi, are derived from the primitive endodermal cells that line the embryonic outpouching. See Yesner, supra. During embryonic development, multipotent endodermal stem cells differentiate into many different types of specialized cells, which include ciliated cells for moving inhaled particles, goblet cells for producing mucus, Kulchitsky's cells for endocrine function, and Clara cells and type II pneumocytes for secreting surfactant protein. Id. Improper development and differentiation may cause respiratory disorders and distress in infants, particularly in premature infants, whose lungs cannot produce sufficient surfactant when they are born. Further, some lung cancer cells, particularly small cell carcinomas, are plastic and can alter their phenotype into a number of cell types, including large cell carcinoma, adenocarcinoma and squamous cell carcinoma. Id. Thus, a better understanding of lung development and differentiation may help facilitate understanding of lung cancer initiation and progression.

The most common screening tests for lung cancer are chest x-ray and sputum cytology. Randomized controlled trials have not demonstrated a reduction in lung cancer mortality resulting from screening with chest x-ray and/or sputum cytology. Additionally, sputum cytology has not been shown to be effective when used as an adjunct to annual chest x-ray. Screening with chest x-ray plus sputum cytology appears to detect lung cancer at an earlier stage, but this would be expected in a screening test whether or not it was effective at reducing mortality. Since early detection by current screening methods fails to reduce mortality in lung cancer patients, current lung cancer screening methods are inadequate.

There are two important potential hazards associated with chest radiography screening. First, false positive test results can lead to an unnecessary invasive procedure, such as percutaneous needle biopsy or thoracotomy. These procedures are costly and due to their invasive nature carry risks of their own. The second hazard with chest radiography screening is overdiagnosis. Overdiagnosis is the diagnosis of a small or slowly growing tumor that would not have become clinically significant had it not been detected by screening. Although overdiagnosis is almost impossible to document in a living individual, autopsy studies suggest that many individuals die with lung cancer rather than from it.

Additionally, the spectrum of lung cancer type has shifted over the last two decades. Whereas the most common type used to be squamous cell cancer (usually centrally located), the most common type now is adenocarcinoma (usually peripherally located). The latter may be more amenable to early detection by chest x-ray, the limitations of which are described above. In contrast, sputum cytology, is more sensitive in the detection of squamous cell cancer than in detecting adenocarcinoma, and therefore lacks usefulness in detecting the more common adenocarcinomas. Clearly, new highly sensitive non-invasive methods of detecting lung cancer are needed.

There are intensive efforts to improve lung cancer screening with newer technologies, including low-dose helical computed tomography (LDCT) and molecular techniques. LDCT is far more sensitive than chest radiography. In a recent screening study, CT detected almost 6 times as many stage I lung cancers as chest radiography and most of these tumors were 1 cm or less in diameter. However, the effectiveness of screening with LDCT has not yet been evaluated in a controlled clinical trial.

There are two potential hazards that must be considered against any potential benefit of screening with LDCT. The more common and familiar hazard is the false positive test result, which may lead to anxiety and invasive diagnostic procedures. A less familiar hazard is overdiagnosis, the diagnosis of a condition that would not have become clinically significant had it not been detected by screening. In the case of screening with LDCT, overdiagnosis could lead to unnecessary diagnosis of lung cancer requiring some combination of surgery, e.g., lobectomy, chemotherapy and radiation therapy. As stated above, overdiagnosis is almost impossible to document in a living individual. In one large study, about one-sixth of all lung cancers found at autopsy had not been clinically recognized before death. Furthermore, autopsy probably fails to detect many small lung cancers that are detectable by CT.

Current therapies for lung cancer are quite limited. Generally, patient options comprise surgery, radiation therapy, and chemotherapy.

Depending on the type and stage of a lung cancer, surgery may be used to remove the tumor along with some surrounding lung tissue. A lobectomy refers to a lobe (section) of the lung being removed. If the entire lung is removed, the surgery is called a pneumonectomy. Removing only part of a lobe is known as a segmentectomy or wedge resection.

If the cancer has spread to the brain, benefit may be gained from removal of the brain metastasis. This involves a craniotomy (surgery through a hole in the skull).

For radiation therapy several methods exist. External beam radiation therapy uses radiation delivered from outside the body that is focused on the cancer. This type of radiation therapy is most often used to treat a primary lung cancer or its metastases to other organs.

Brachytherapy uses a small pellet of radioactive material placed directly into the cancerous tissue or into the airway next to the cancer. Radiation therapy is sometimes used as the main (primary) treatment of lung cancer, especially if the general health of the patient is too poor to undergo surgery. Brachytherapy can also be used to help relieve blockage of large airways by cancer.

Additionally, radiation therapy can be used as a post surgical treatment to kill very small deposits of cancer that cannot be seen or removed during surgery. Radiation therapy can also be used to palliate (relieve) symptoms of lung cancer such as pain, bleeding, difficulty swallowing, and problems caused by brain metastases.

For chemotherapy, cisplatin or a related drug, carboplatin, are the chemotherapy agents most often used in treating NSCLC. Recent studies found that combining either of these with drugs such as gemcitabine, paclitaxel, docetaxel, etoposide, or vinorelbine appear to be more effective in treating NSCLC.

Recently, the National Comprehensive Cancer Network (NCCN; nccn with the extension org of the world wide web), an alliance of nineteen of the world's leading cancer centers, announces a major update of the NCCN Non-Small Cell Lung Cancer Clinical Practice Guidelines. The NCCN is widely recognized as a standard for clinical policy in oncology.

Recently approved targeted therapy, gefitinib (Iressa®, AstraZeneca Pharmaceuticals LP) is now recommended as third-line therapy and as second-line only if the platinum/docetaxel combination was used as first-line therapy.

The NCCN's Non-Small Cell Lung Cancer (NSCLC) guidelines contain recommendations for administration of chemotherapy to patients with this disease including patient selection criteria and definition of first-, second-, and third-line agents and combinations.

Chemotherapeutic agents are specified as two-agent regimens for first-line therapy, two agent regimens or single agents for second-line therapy, and one single agent for third-line therapy. Agents used in first- and second-line therapy are: cisplatin (Platinol®, Bristol-Myers Squibb Company), carboplatin (Paraplatin®, Bristol-Myers Squibb Company), paclitaxel (Taxol®, Bristol-Myers Squibb Company), docetaxel (Taxotere®, Aventis Pharmaceuticals Inc.), vinorelbine (Navelbine®, GlaxoSmithKline), gemcitabine (Gemzar®, Eli Lilly and Company), etoposide (Toposar®, Pfizer, Inc.; VePesid®, Bristol-Myers Squibb Company; Etopophos®, Bristol-Myers Squibb Company), irinotecan (Camptosar®, Pfizer, Inc.), vinblastine (Velban®, Eli Lilly and Company), mitomycin (Mutamycin®, Bristol-Myers Squibb Company), and ifosfamide (Ifex®, Bristol-Myers Squibb Company).

Some of the usual chemotherapy combinations used for patients with SCLC include: EP (etoposide and cisplatin); ET (etoposide and carboplatin); ICE (ifosfamide, carboplatin, and etoposide); and CAV (cyclophosphamide, doxorubicin, and vincristine).

New drugs such as gemcitabine, paclitaxel, vinorelbine, topotecan, and teniposide have shown promising results in some SCLC studies. Growth factors may be given in conjunction to chemotherapy agents if patient health is good. The administration of growth factors help prevent bone marrow side effects.

Ongoing or recently completed therapeutic trials for various compounds to treat lung cancer include alitretinoin (Panretin®, Ligand Pharmaceuticals), topotecan HCl (Hycamtin® GlaxoSmithKline), liposomal ether lipid (Elan Pharmaceutical), cantuzumab mertansine (ImmunoGen), Gavax® (Cell Genesys), vincristine (Onco TCS®, Inex Pharmaceuticals), Neovastat® (AEterna Laboratories), squalamine (Genaera), mirostipen (Human Genome Sciences Inc.), Advexin® (Introgen Therapeutics), biricodar dicitrate (Incel®, Vertex Pharmaceuticals), flavopiridol (Aventis), Affintac® (Eli Lilly and Company), pivaloyloxymethylbutyrate (Pivanex®, Titan Pharmaceuticals), tirapazamine (Tirazone®, Sanofi-Synthelabo Pharmaceuticals), irinotecan (Camptosar®, Pharmacia), tezacitabine (Chiron), cisplatin/vinblastine/amifostine (MedhImnune), paclitaxel/carboplatin/amifostine (MedImmune), Oncomyc-NG® (AVI BioPharma), exisulind/vinorelbine (Aptosyn®/Navelbine®, Cell Pathyways), tariquidar (QLT), Xyotax® (Cell Therapeutics), PEG-camptothecin (Prothecan®, Enzon), decitabine (SuperGen), Tarceva® (OSI Pharmaceuticals), ABX-EGF (Abgenix), Tocosol Paclitaxel® (Sonus Pharmaceuticals), TheraFab® (Antisoma), minodronate (Yamanouchi Pharmaceutical), exisulind/docetaxel/carboplatin (Aptosyn®/Taxotere®/Paraplatin®, Cell Pathways), exisulind/gemcitabine HCl (Aptosyn®/Gemzar®, Cell Pathways), IMC-C225/carboplatin/paclitaxel (Erbitux®/carboplatin®/paclitaxel®, InClone Systems), and vinorelbine (Navelbine®, GlaxoSmithKline).

As indicated above, many therapeutics are recommended for use in combination as a first-line therapy or only if other therapeutics have failed as second-, and third-line agents. While there are many compounds in ongoing or recently completed therapeutic trials, there is great need for additional therapeutic compounds capable of treating early stage and advanced or metastasized lung cancer.

Accordingly, there is a great need for more sensitive and accurate methods for predicting whether a person is likely to develop lung cancer, for diagnosing lung cancer, for monitoring the progression of the disease, for staging the lung cancer, for determining whether the lung cancer has metastasized and for imaging the lung cancer. There is also a need for better treatment of lung cancer. Further, there is a great need for diagnosing and treating noncancerous lung disorders such as emphysema, pneumonia, lung infection, pulmonary fibrosis, cystic fibrosis and asthma. There is also a need for compositions and methods of using these compositions to identify lung tissue for forensic purposes and for determining whether a particular cell or tissue exhibits lung-specific characteristics.

As discussed above, each of the methods for diagnosing and staging lung is limited by the technology employed. Accordingly, there is need for sensitive molecular and cellular markers for the detection of ovarian, pancreatic, lung or breast cancer. There is a need for molecular markers for the accurate staging, including clinical and pathological staging, of lung to optimize treatment methods. Finally, there is a need for sensitive molecular and cellular markers to monitor the progress of cancer treatments, including markers that can detect recurrence of lung following remission.

The present invention provides alternative methods of treating ovarian, pancreatic, lung or breast cancer that overcome the limitations of conventional therapeutic methods as well as offer additional advantages that will be apparent from the detailed description below.

### SUMMARY OF THE INVENTION

This invention is directed to an isolated Lng105 antibody that internalizes upon binding to Lng105 on a mammalian cell in vivo. In one embodiment, the antibody is a monoclonal antibody. In an alternative embodiment, the antibody is an antibody fragment or a chimeric or a humanized antibody. The monoclonal antibody may be produced by a hybridoma selected from the group of hybridomas deposited under American Type Culture Collection accession number PTA-5878, PTA-5879, PTA-6146, PTA-6147 and PTA-6629. The antibody may compete for binding to the same epitope as the epitope bound by the monoclonal antibody produced by a hybridoma selected from the group of hybridomas deposited under the American Type Culture Collection accession number PTA-5878, PTA-5879, PTA-6146, PTA-6147 and PTA-6629.

The invention is also directed to conjugated antibodies. They may be conjugated to a growth inhibitory agent or a cytotoxic agent. The cytotoxic agent may be selected from the group consisting of toxins, antibiotics, radioactive isotopes and nucleolytic enzymes or a toxin. Examples of toxins include, but are not limited to, maytansin, maytansinoids, saporin, gelonin, ricin or calicheamicin

In one embodiment, the mammalian cell is a cancer cell. Preferably, the anti-Lng105 monoclonal antibody that inhibits the growth of Lng105-expressing cancer cells in vivo.

The antibody may be produced in bacteria. Alternatively, the antibody may be a humanized form of an anti-Lng105 antibody produced by a hybridoma selected from the group of hybridomas having ATCC accession number PTA-5878, PTA-5879, PTA-6146, PTA-6147 and PTA-6629. Preferably, the a cancer selected from the group consisting of ovarian, pancreatic, lung or breast cancer. The invention is also directed to a method of producing the antibodies comprising culturing an appropriate cell and recovering the antibody from the cell culture.

The invention is also directed to compositions comprising the antibodies and a carrier. In one embodiment, the antibody is conjugated to a cytotoxic agent. The cytotoxic agent is a radioactive isotope.

The invention is also directed to a method of killing and/or detecting an Lng105-expressing cancer cell, comprising contacting the cancer cell with the antibodies of this invention, thereby killing and/or detecting the cancer cell. The cancer cell may be selected from the group consisting of ovarian, pancreatic, lung or breast cancer cell.

The ovarian, or breast cancer may be ovarian serous adenocarcinoma or breast infiltrating ductal carcinoma or metastatic cancer. The invention is also directed to a method of alleviating an Lng105-expressing cancer in a mammal, comprising administering a therapeutically effective amount of the antibodies to the mammal.

In addition, the invention is directed to an article of manufacture comprising a container and a composition contained therein, wherein the composition comprises an antibody as described herein and further comprising a package insert indicating that the composition can be used to treat ovarian, pancreatic, lung or breast cancer.

### BRIEF DESCRIPTION OF THE FIGURES

FIG. 1 shows the Lng105 epitope map for anti-Lng105 antibodies.

FIG. 2 shows Lng105 serum levels in individuals with lung cancer.

FIG. 3 shows Lng105 serum levels in individuals with benign lung diseases.

FIG. 4 shows Lng105 serum levels in in individuals non-lung benign diseases.

FIG. 5 shows Lng105 serum levels in various stages of lung cancer.

FIG. 6 shows Lng105 serum levels in various lung cancer histopathologic types.

FIG. 7 shows Lng105 serum levels in individuals with lung cancer.

FIG. 8 shows ROC Analysis of Lng105 in lung cancer vs normal and benign lung disease samples.

FIG. 9 shows ROC Analysis of Lng105 in lung cancer vs normal, benign lung disease and non-lung cancer samples.

FIG. 10 shows ROC analysis of Lng105 and CA125 in lung cancer vs normal, benign lung disease and non-lung cancer samples.

### DETAILED DESCRIPTION OF THE INVENTION

Definitions and General Techniques

Human “Lng105 ” as used herein, refers to a protein of 420 amino acids that is secreted, whose nucleotide and amino acid sequence sequences are as disclosed in e.g., WO 99/60160 DIADEXUS, Human lung specific gene Lng105; WO9822597; WO9936550; WO200004137; WO9813484; WO9811236 the disclosures of which are hereby expressly incorporated by reference.

Lng105 has also been identified as Napsin A, an aspartic proteinase. see: RefSeq IDs: NM_{—}004851 and NP_{—}004842; The RefSeq database annotates Napsin A as:
The activation peptides of aspartic proteinases plays role as inhibitors of the active site. These peptide segments, or pro-parts, are deemed important for correct folding, targeting, and control of the activation of aspartic proteinase zymogens. The pronapsin A gene is expressed predominantly in lung and kidney. Its translation product is predicted to be a fully functional, glycosylated aspartic proteinase precursor containing an RGD motif and an additional 18 residues at its C-terminus.

Additionally, other publications have characterized Napsin A (aka TA02) and discussed its association with cancer. See: Koelsch, G. et al Multiple functions of pro-parts of aspartic proteinase zymogens. FEBS Lett. 343:6-10 (1994); Blundell, T. L. et al. The aspartic proteinases. An historical overview. Adv. Exp. Med. Biol. 436:1-13 (1998); Tatnell, P. J. et al. Napsins: new human aspartic proteinases. Distinction between two closely related genes. FEBS Lett. 441:43-48 (1998); Yan, R. et al. Membrane-anchored aspartyl protease with Alzheimer's disease beta-secretase activity. Nature 402:533-537 (1999); Schauer-Vukasinovic et al., Detection of immunoreactive napsin A in human urine. Biocehm. et Biosphy Acta. 1524:51-56 (2000); Cook, M. et al. Pronapsin A and B gene expression in normal and malignant human lung and mononuclear blood cells. Biochim. Biophys. Acta 1577:10-16 (2002); Ueno, T. et al., Aspartic proteinase napsin is a useful marker for diagnosis of primary lung adenocarcinoma. Br. J Caizcer 88 (8), 1229-1233 (2003); Brasch, F., et al., Involvement of napsin A in the C- and N-terminal processing of surfactant protein B in type-II pneumocytes of the human lung. JBC 278 (49), 49006-49014 (2003); Ota, T. et al. Complete sequencing and characterization of 21,243 full-length human cDNAs. Nat. Genet. 36:40-45 (2004) the disclosures of which are hereby expressly incorporated by reference. While Lng105/napsin A has been described in the above references, we believe this is the first time antibodies specific to Lng105 have been generated against a full length Lng105 protein (non-peptide) and used in an ELISA to detect Lng105 in a bodily fluid to determine the presence of cancer. Lng105 as used herein include pre- and pro-forms, allelic variants and conservative substitution mutants of a protein which has Lng105 biological activity, specifically aspartic proteinase activity.

The term “bodily fluid” as used herein includes any fluid like subatance derived from the body selected from the group comprising, blood (whole blood, plasma, serum or other sub-fraction), semen, tears, feces, urine, vomitus, respiratory secretions (such as nasal discharge and saliva), cerebrospinal fluid, lymphatic fluid, synovial fluid, sweat, chyle, gastric, pancreatic, and intestinal juices, aqueous humor and drainage from scrapes and cuts.

The term “antibody” (Ab) as used herein includes monoclonal antibodies, polyclonal antibodies, multispecific antibodies (e.g. bispecific antibodies), and antibody fragments, so long as they exhibit the desired biological activity. The term “immunoglobulin” (Ig) is used interchangeably with “antibody” herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for [L and F isotypes. Each 6 L chain has at the N-terminus, a variable domain (VL) followed by a constant domain (CL) at its other end.

The VL is aligned with the VH and the CL is aligned with the first constant domain of the heavy chain (CHI).

Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Teff and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are farther divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 1-10-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a P-sheet configuration, connected by three hypervariable regions, which form loops connecting, and in some cases forming part of, the P-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (LI), 5056 (L2) and 89-97 (L3) in the VL, and around about 1-35 (HI), 50-65 (H2) and 95-102 (113) in the VH; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (LI), 50-52 (L2) and 91-96 (U) in the VL, and 26-32 (HI), 53-55 (1-12) and 96-101 (H3) in the VH; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a CL and at least heavy chain constant domains, CHI, CH2 and CH3. The constant domains may be native sequence constant domains (e.g. human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CHI). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of 8 Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

A “native sequence” polypeptide is one which has the same amino acid sequence as a polypeptide (e.g., antibody) derived from nature. Such native sequence polypeptides can be isolated from nature or can be produced by recombinant or synthetic means. Thus, a native sequence polypeptide can have the amino acid sequence of a naturally occurring human polypeptide, murine polypeptide, or polypeptide from any other mammalian species.

The term “amino acid sequence variant” refers to a polypeptide that has amino acid sequences that differ to some extent from a native sequence polypeptide. Ordinarily, amino acid sequence variants of Lng105 will possess at least about 70% homology with the native sequence Lng105, preferably, at least about 80%, more preferably at least about 85%, even more preferably at least about 90% homology, and most preferably at least 95%. The amino acid sequence variants can possess substitutions, deletions, and/or insertions at certain positions within the amino acid sequence of the native amino acid sequence.

The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcFRI.

“Homology” is defined as the percentage of residues in the amino acid sequence variant that are identical after aligning the sequences and introducing gaps, if necessary, to achieve the maximum percent homology. Methods and computer programs for the alignment are well known in the art. Sequence similarity may be measured by any common sequence analysis algorithm, such as GAP or BESTFIT or other variation Smith-Waterman alignment. See, T. F. Smith and M. S. Waterman, J. Mol. Biol. 147:195-197 (1981) and W. R. Pearson, Genomics 11:635-650 (1991).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, an anti-Lng105 antibody that “internalizes” is one that is taken up by (i.e., enters) the cell upon binding to Lng105 on a mammalian cell (i.e. cell surface Lng105). The internalizing antibody will of course include antibody fragments, human or humanized antibody and antibody conjugate. For therapeutic applications, internalization in vivo is contemplated. The number of antibody molecules internalized will be sufficient or adequate to kill an Lng105-expressing cell, especially an Lng105-expressing cancer cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the tumor cell.

Whether an anti-Lng105 antibody internalizes upon binding Lng105 on a mammalian cell can be determined by various assays including those described in the experimental examples below. For example, to test internalization in vivo, the test antibody is labeled and introduced into an animal known to have Lng105 expressed on the surface of certain cells. The antibody can be radiolabeled or labeled with fluorescent or gold particles, for instance. Animals suitable for this assay include a mammal such as a NCR nude mouse that contains a human Lng105-expressing tumor transplant or xenograft, or a mouse into which cells transfected with human Lng105 have been introduced, or a transgenic mouse expressing the human Lng105 transgene. Appropriate controls include animals that did not receive the test antibody or that received an unrelated antibody, and animals that received an antibody to another antigen on the cells of interest, which antibody is known to be internalized upon binding to the antigen. The antibody can be administered to the animal, e.g., by intravenous injection. At suitable time intervals, tissue sections of the animal can be prepared using known methods or as described in the experimental examples below, and analyzed by light microscopy or electron microscopy, for internalization as well as the location of the internalized antibody in the cell. For internalization in vitro, the cells can be incubated in tissue culture dishes in the presence or absence of the relevant antibodies added to the culture media and processed for microscopic analysis at desired time points. The presence of an internalized, labeled antibody in the cells can be directly visualized by microscopy or by autoradiography if radiolabeled antibody is used. Alternatively, in a quantitative biochemical assay, a population of cells comprising Lng105-expressing cells are contacted in vitro or in vivo with a radiolabeled test antibody and the cells (if contacted in vivo, cells are then isolated after a suitable amount of time) are treated with a protease or subjected to an acid wash to remove uninternalized antibody on the cell surface. The cells are ground up and the amount of protease resistant, radioactive counts per minute (cpm) associated with each batch of cells is measured by passing the homogenate through a scintillation counter. Based on the known specific activity of the radiolabeled antibody, the number of antibody molecules internalized per cell can be deduced from the scintillation counts of the ground-up cells. Cells are “contacted” with antibody in vitro preferably in solution form such as by adding the cells to the cell culture media in the culture dish or flask and mixing the antibody well with the media to ensure uniform exposure of the cells to the antibody. Instead of adding to the culture media, the cells can be contacted with the test antibody in an isotonic solution such as PBS in a test tube for the desired time period. In vivo, the cells are contacted with antibody by any suitable method of administering the test antibody such as the methods of administration described below when administered to a patient.

The faster the rate of internalization of the antibody upon binding to the Lng105-expressing cell in vivo, the faster the desired killing or growth inhibitory effect on the target Lng105-expressing cell can be achieved, e.g., by a cytotoxic immunoconjugate. Preferably, the kinetics of internalization of the anti-Lng105 antibodies are such that they favor rapid killing of the Lng105-expressing target cell. Therefore, it is desirable that the anti-Lng105 antibody exhibit a rapid rate of internalization preferably, within 24 hours from administration of the antibody in vivo, more preferably within about 12 hours, even more preferably within about 30 minutes to 1 hour, and most preferably, within about 30 minutes. The present invention provides antibodies that internalize as fast as about 15 minutes from the time of introducing the anti-Lng105 antibody in vivo. The antibody will preferably be internalized into the cell within a few hours upon binding to Lng105 on the cell surface, preferably within 1 hour, even more preferably within 15-30 minutes.

To determine if a test antibody can compete for binding to the same epitope as the epitope bound by the anti-Lng105 antibodies of the present invention including the antibodies produced by the hybridomas deposited with the ATCC, a cross-blocking assay e.g., a competitive ELISA assay can be performed. In an exemplary competitive ELISA assay, Lng105 coated on the wells of a microtiter plate is pre-incubated with or without candidate competing antibody and then the biotin-labeled anti-Lng105 antibody of the invention is added.

The amount of labeled anti-Lng105 antibody bound to the Lng105 antigen in the wells is measured using avidin-peroxidase conjugate and appropriate substrate. The antibody can be labeled with a radioactive or fluorescent label or some other detectable and measurable label. The amount of labeled anti-Lng105 antibody that bound to the antigen will have an indirect correlation to the ability of the candidate competing antibody (test antibody) to compete for binding to the same epitope, i.e., the greater the affinity of the test antibody for the same epitope, the less labeled antibody will be bound to the antigen-coated wells. A candidate competing antibody is considered an antibody that binds substantially to the same epitope or that competes for binding to the same epitope as an antiLng105 antibody of the invention if the candidate antibody can block binding of the Lng105 antibody by at least 20%, preferably by at least 20-50%, even more preferably, by at least 50% as compared to the control performed in parallel in the absence of the candidate competing antibody (but may be in the presence of a known noncompeting antibody). It will be understood that variations of this assay can be performed to arrive at the same quantitative value.

An antibody having a “biological characteristic” of a designated antibody, such as any of the monoclonal antibodies Lng105.D3, Lng105.D4, Lng105.D6, Lng105.D7, Lng105.D 1, Lng105.D12, Lng105.D13, Lng105.D14, Lng105.D16, Lng105.D17, Lng105.D18, Lng105.D19, Lng105.D20, Lng105.D22, Lng105.D27, Lng105.D28, Lng105.D31, Lng105.D32, Lng105.D36, Lng105.D37, Lng105.D39, Lng105.D40, Lng105.D42, Lng105.D44, Lng105.D45, Lng105.D47, Lng105.D48, Lng105.D49, Lng105.D51, Lng105.D53, Lng105.D54, Lng105.D63, Lng105.D65, Lng105.D71, Lng105.D73, Lng105.D74, Lng105.D79, Lng105.J3, Lng105.J6, Lng105.J10, Lng105.J13, Lng105.J15, Lng105.J16, Lng105.J21, Lng105.J23, Lng105.J25, Lng105.J31, Lng105.J32, Lng105.J41, Lng105.J43, Lng105.J50, Lng105.J54, Lng105.J57, Lng105.J64, Lng105.J71, Lng105.J85, Lng105.J86, Lng105.J87, Lng105.J88, Lng105.J91, Lng105.J93, Lng105.J96, Lng105.J99, Lng105.J100, Lng105.J101, Lng105.J104, Lng105.J105, Lng105.J106, Lng105.J109, Lng105.J111, Lng105.J112, Lng105.J114, Lng105.J115, Lng105.J237, Lng105.J238, Lng105.J250, Lng105.J255, Lng105.J263, Lng105.J264, Lng105.J265, Lng105.J276 and Lng105.J281, is one which possesses one or more of the biological characteristics of that antibody which distinguish it from other antibodies that bind to the same antigen, Lng105. For example, an antibody with a biological characteristic of Lng105.D3, Lng105.D4, Lng105.D6, Lng105.D7, Lng105.D11, Lng105.D12, Lng105.D13, Lng105.D14, Lng105.D16, Lng105.D17, Lng105.D18, Lng105.D19, Lng105.D20, Lng105.D22, Lng105.D27, Lng105.D28, Lng105.D31, Lng105.D32, Lng105.D36, Lng105.D37, Lng105.D39, Lng105.D40, Lng105.D42, Lng105.D44, Lng105.D45, Lng105.D47, Lng105.D48, Lng105.D49, Lng105.D51, Lng105.D53, Lng105.D54, Lng105.D63, Lng105.D65, Lng105.D71, Lng105.D73, Lng105.D74, Lng105.D79, Lng105.J3, Lng105.J6, Lng105.J10, Lng105.J13, Lng105.J15, Lng105.J16, Lng105.J21, Lng105.J23, Lng105.J25, Lng105.J31, Lng105.J32, Lng105.J41, Lng105.J43, Lng105.J50, Lng105.J54, Lng105.J57, Lng105.J64, Lng105.J71, Lng105.J85, Lng105.J86, Lng105.J87, Lng105.J88, Lng105.J91, Lng105.J93, Lng105.J96, Lng105.J99, Lng105.J100, Lng105.J101, Lng105.J104, Lng105.J105, Lng105.J106, Lng105.J109, Lng105.J111, Lng105.J112, Lng105.J114, Lng105.J115, Lng105.J237, Lng105.J238, Lng105.J250, Lng105.J255, Lng105.J263, Lng105.J264, Lng105.J265, Lng105.J276 and Lng105.J281 (e.g. which competes for binding or blocks binding of monoclonal antibody Lng105.D3, Lng105.D4, Lng105.D6, Lng105.D7, Lng105.D11, Lng105.D12, Lng105.D13, Lng105.D14, Lng105.D16, Lng105.D17, Lng105.D18, Lng105.D19, Lng105.D20, Lng105.D22, Lng105.D27, Lng105.D28, Lng105.D31, Lng105.D32, Lng105.D36, Lng105.D37, Lng105.D39, Lng105.D40, Lng105.D42, Lng105.D44, Lng105.D45, Lng105.D47, Lng105.D48, Lng105.D49, Lng105.D51, Lng105.D53, Lng105.D54, Lng105.D63, Lng105.D65, Lng105.D71, Lng105.D73, Lng105.D74, Lng105.D79, Lng105.J3, Lng105.J6, Lng105.J10, Lng105.J13, Lng105.J15, Lng105.J16, Lng105.J21, Lng105.J23, Lng105.J25, Lng105.J31, Lng105.J32, Lng105.J41, Lng105.J43, Lng105.J50, Lng105.J54, Lng105.J57, Lng105.J64, Lng105.J71, Lng105.J85, Lng105.J86, Lng105.J87, Lng105.J88, Lng105.J91, Lng105.J93, Lng105.J96, Lng105.J99, Lng105.J100, Lng105.J101, Lng105.J104, Lng105.J105, Lng105.J106, Lng105.J109, Lng105.J111, Lng105.J112, Lng105.J114, Lng105.J115, Lng105.J237, Lng105.J238, Lng105.J250, Lng105.J255, Lng105.J263, Lng105.J264, Lng105.J265, Lng105.J276 and Lng105.J281 to Lng105), be able to target an Lng105-expressing tumor cell in vivo and will internalize upon binding to Lng105 on a mammalian cell in vivo. Likewise, an antibody with the biological characteristic of the Lng105.D3, Lng105.D4, Lng105.D6, Lng105.D7, Lng105.D11, Lng105.D12, Lng105.D13, Lng105.D14, Lng105.D16, Lng105.D17, Lng105.D18, Lng105.D19, Lng105.D20, Lng105.D22, Lng105.D27, Lng105.D28, Lng105.D31, Lng105.D32, Lng105.D36, Lng105.D37, Lng105.D39, Lng105.D40, Lng105.D42, Lng105.D44, Lng105.D45, Lng105.D47, Lng105.D48, Lng105.D49, Lng105.D51, Lng105.D53, Lng105.D54, Lng105.D63, Lng105.D65, Lng105.D71, Lng105.D73, Lng105.D74, Lng105.D79, Lng105.J3, Lng105.J6, Lng105.J10, Lng105.J13, Lng105.J15, Lng105.J16, Lng105.J21, Lng105.J23, Lng105.J25, Lng105.J31, Lng105.J32, Lng105.J41, Lng105.J43, Lng105.J50, Lng105.J54, Lng105.J57, Lng105.J64, Lng105.J71, Lng105.J85, Lng105.J86, Lng105.J87, Lng105.J88, Lng105.J91, Lng105.J93, Lng105.J96, Lng105.J99, Lng105.J100, Lng105.J101, Lng105.J104, Lng105.J105, Lng105.J106, Lng105.J109, Lng105.J111, Lng105.J112, Lng105.J114, Lng105.J115, Lng105.J237, Lng105.J238, Lng105.J250, Lng105.J255, Lng105.J263, Lng105.J264, Lng105.J265, Lng105.J276 and Lng105.J281 antibody will have the same epitope binding, targeting, internalizing, tumor growth inhibitory and cytotoxic properties of the antibody.

The term “antagonist” antibody is used in the broadest sense, and includes an antibody that partially or fully blocks, inhibits, or neutralizes a biological activity of a native Lng105 protein disclosed herein. Methods for identifying antagonists of an Lng105 polypeptide may comprise contacting an Lng105 polypeptide or a cell expressing Lng105 on the cell surface, with a candidate antagonist antibody and measuring a detectable change in one or more biological activities normally associated with the Lng105 polypeptide.

An “antibody that inhibits the growth of tumor cells expressing Lng105” or a “growth inhibitory” antibody is one which binds to and results in measurable growth inhibition of cancer cells expressing or overexpressing Lng105. Preferred growth inhibitory anti-Lng105 antibodies inhibit growth of Lng105-expressing tumor cells e.g., ovarian, pancreatic, lung or breast cancer cells) by greater than 20%, preferably from about 20% to about 50%, and even more preferably, by greater than 50% (e.g. from about 50% to about 100%) as compared to the appropriate control, the control typically being tumor cells not treated with the antibody being tested. Growth inhibition can be measured at an antibody concentration of about 0.1 to 30 pg/ml or about 0.5 nM to 200 nM in cell culture, where the growth inhibition is determined 1-10 days after exposure of the tumor cells to the antibody. Growth inhibition of tumor cells in vivo can be determined in various ways such as is described in the Experimental Examples section below. The antibody is growth inhibitory in vivo if administration of the anti-Lng105 antibody at about 1 pg/kg to about 100 mg/kg body weight results in reduction in tumor size or tumor cell proliferation within about 5 days to 3 months from the first administration of the antibody, preferably within about 5 to 30 days.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses Lng105. Preferably the cell is a tumor cell, e.g. an ovarian, pancreatic, lung or breast cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody which induces apoptosis is one which results in about 2 to 50 fold, preferably about 5 to 50 fold, and most preferably about 10 to 50 fold, induction of amiexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigenbearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamnna receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIEB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126.330-41 (1995). Other FcRs, including those to be identified in the future, are encompassed by the term “FcR” herein. The term also includes the neonatal receptor, FcRn, which is responsible for the transfer, of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g. from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996) may be performed.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

A “Lng105-expressing cell” is a cell which expresses endogenous or transfected Lng105 on the cell surface. A “Lng105-expressing cancer” is a cancer comprising cells that have Lng105 protein present on the cell surface. A “Lng105-expressing cancer” produces sufficient levels of Lng105 on the surface of cells thereof, such that an anti-Lng105 antibody can bind thereto and have a therapeutic effect with respect to the cancer. A cancer which “overexpresses” Lng105 is one which has significantly higher levels of Lng105.At the cell surface thereof, compared to a noncancerous cell of the same tissue type. Such overexpression may be caused by gene amplification or by increased transcription or translation. Lng105 overexpression may be determined in a diagnostic or prognostic assay by evaluating increased levels of the Lng105 protein present on the surface of a cell (e.g. via an immunohistochemistry assay; FACS analysis). Alternatively, or additionally, one may measure levels of Lng105-encoding nucleic acid or MRNA in the cell, e.g. via fluorescent in situ hybridization; (FISH; see WO98/45479 published October, 1998), Southern blotting, Northern blotting, or polymerase chain reaction (PCR) techniques, such as real time quantitative PCR (RT-PCR). One may also study Lng105 overexpression by measuring shed antigen in a biological fluid such as serum, e.g., using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody. An Lng105-expressing cancer includes ovarian, pancreatic, lung or breast cancer.

A “mammal” for purposes of treating a cancer or alleviating the symptoms of cancer, refers to any mammal, including-humans, domestic and farm animals, and zoo, sports, or pet animals, such as dogs, cats, cattle, horses, sheep, pigs, goats, rabbits, etc. Preferably, the mammal is human.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for an Lng105-expressing cancer if, after receiving a therapeutic amount of an anti-Lng105 antibody according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the anti-Lng105 antibody may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR).

The term “therapeutically effective amount” refers to an amount of an antibody or a drug effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”. To the extent the drug may prevent growth and/or kill existing cancer cells, it may be cytostatic and/or cytotoxic.

“Chronic” administration refers to administration of the agent(s) in a continuous mode as opposed to an acute mode, so as to maintain the initial therapeutic effect (activity) for an extended period of time.

“Intermittent” administration is treatment that is not consecutively done without interruption, but rather is cyclic in nature.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed.

Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, malunose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, SM¹⁵³, Bi²¹², P³², and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, e.g., gelonin, ricin, saporin, and the various antitumor or anticancer agents disclosed below. Other cytotoxic agents are described below. A tumoricidal agent causes destruction of tumor cells.

A “growth inhibitory agent” when used herein refers to a compound or composition which inhibits growth of a cell, especially an Lng105-expressing cancer cell, either in vitro or in vivo. Thus, the growth inhibitory agent may be one which significantly reduces the percentage of Lng105-expressing cells in S phase. Examples of growth inhibitory agents include agents that block cell cycle progression (at a place other than S phase), such as agents that induce GI arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest GI also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Label” as used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

The term “epitope tagged” used herein refers to a chimeric polypeptide comprising an anti-Lng105 antibody polypeptide fused to a “tag polypeptide”. The tag polypeptide has enough residues to provide an epitope against which an antibody can be made, yet is short enough such that it does not interfere with activity of the Ig polypeptide to which it is fused. The tag polypeptide is also preferably fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

A “small molecule” is defined herein to have a molecular weight below about 500 Daltons.

The term “package insert” is used to refer to instructions customarily included in commercial packages of therapeutic products, that contain information about the indications, usage, dosage, administration, contraindications and/or warnings concerning the use of such therapeutic products.

An “isolated nucleic acid” is a nucleic acid, e.g., an RNA, DNA, or a mixed polymer, which is substantially separated from other genome DNA sequences as well as proteins or complexes such as ribosomes and polymerases, which naturally accompany a native sequence. The term embraces a nucleic acid sequence which has been removed from its naturally occurring environment, and includes recombinant or cloned DNA isolates and chemically synthesized analogues or analogues biologically synthesized by heterologous systems. A substantially pure molecule includes isolated forms of the molecule.

“Vector” includes shuttle and expression vectors. Typically, the plasmid construct will also include an origin of replication (e.g., the Co1E1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

The cell that produces an anti-Lng105 antibody of the invention will include the parent hybridoma cell e.g., the hybridomas that are deposited with the ATCC, as well as bacterial and eukaryotic host cells into which nucleic acid encoding the antibodies have been introduced. Suitable host cells are disclosed below.

RNA interference refers to the process of sequence-specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877) has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 both suggest that siRNA “may include modifications to either the phosphate-sugar back bone or the nucleoside to include at least one of a nitrogen or sulfur heteroatom”, however neither application teaches to what extent these modifications are tolerated in siRNA molecules nor provide any examples of such modified siRNA. Kreutzer and Limmer, Canadian Patent Application No. 2,359,180, also describe certain chemical modifications for use in dsRNA constructs in order to counteract activation of double stranded-RNA-dependent protein kinase PKR, specifically 2′-amino or 2′-O-methyl nucleotides, and nucleotides containing a 2′-O or 4′-C methylene bridge. However, Kreutzer and Limmer similarly fail to show to what extent these modifications are tolerated in siRNA molecules nor do they provide any examples of such modified siRNA.

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that “RNAs with two [phosphorothioate] modified bases also had substantial decreases in effectiveness as RNAi triggers (data not shown); [phosphorothioate] modification of more than two residues greatly destabilized the RNAs in vitro and we were not able to assay interference activities.” Id. at 1081. The authors also tested certain modifications at the 2′-position of the nucleotide sugar in the long siRNA transcripts and observed that substituting deoxynucleotides for ribonucleotides “produced a substantial decrease in interference activity”, especially in the case of Uridine to Thymidine and/or Cytidine to deoxy-Cytidine substitutions. Id. In addition, the authors tested certain base modifications, including substituting 4-thiouracil, 5-bromouracil, 5-iodouracil, 3-(aminoallyl)uracil for uracil, and inosine for guanosine in sense and antisense strands of the siRNA, and found that whereas 4-thiouracil and 5-bromouracil were all well tolerated, inosine “produced a substantial decrease in interference activity” when incorporated in either strand. Incorporation of 5-iodouracil and 3-(aminoallyl)uracil in the antisense strand resulted in substantial decrease in RNAi activity as well.

Beach et al., International PCT Publication No. WO 01/68836, describes specific methods for attenuating gene expression using endogenously derived dsRNA. Tuschl et al., International PCT Publication No. WO 01/75164, describes a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due “to the danger of activating interferon response”. Li et al., International PCT Publication No. WO 00/44914, describes the use of specific dsRNAs for use in attenuating the expression of certain target genes. Zernicka-Goetz et al., International PCT Publication No. WO 01/36646, describes certain methods for inhibiting the expression of particular genes in mammalian cells using certain dsRNA molecules. Fire et al., International PCT Publication No. WO 99/32619, describes particular methods for introducing certain dsRNA molecules into cells for use in inhibiting gene expression. Plaetinck et al., International PCT Publication No. WO 00/01846, describes certain methods for identifying specific genes responsible for conferring a particular phenotype in a cell using specific dsRNA molecules. Mello et al., International PCT Publication No. WO 01/29058, describes the identification of specific genes involved in dsRNA mediated RNAi. Deschamps Depaillette et al., International PCT Publication No. WO 99/07409, describes specific compositions consisting of particular dsRNA molecules combined with certain anti-viral agents. Driscoll et al., International PCT Publication No. WO 01/49844, describes specific DNA constructs for use in facilitating gene silencing in targeted organisms. Parrish et al., 2000, Molecular Cell, 6, 1977-1087, describes specific chemically modified siRNA constructs targeting the unc-22 gene of C. elegans. Tuschl et al., International PCT Publication No. WO 02/44321, describe certain synthetic siRNA constructs.

Compositions and Methods of the Invention

The invention provides anti-Lng105 antibodies. In a preferred embodiment, the anti-Lng105 antibodies internalize upon binding to cell surface Lng105 on a mammalian cell. In another preferred embodiment, the anti-Lng105 antibodies destroy or lead to the destruction of tumor cells bearing Lng105.

It was not apparent that Lng105 was internalization-competent. In addition the ability of an antibody to internalize depends on several factors including the affinity, avidity, and isotype of the antibody, and the epitope that it binds. We have demonstrated herein that the cell surface Lng105 is internalization competent upon binding by the anti-Lng105 antibodies of the invention. Additionally, it was demonstrated that the anti-Lng105 antibodies of the present invention can specifically target Lng105-expressing tumor cells in vivo and inhibit or kill these cells. These in vivo tumor targeting, internalization and growth inhibitory properties of the anti-Lng105 antibodies make these antibodies very suitable for therapeutic uses, e.g., in the treatment of various cancers including ovarian, pancreatic, lung or breast cancer. Internalization of the anti-Lng105 antibody is preferred, e.g., if the antibody or antibody conjugate has an intracellular site of action and if the conjugated cytotoxic agent to the antibody does not readily cross the plasma (e.g., the toxin, calicheamicin). Internalization is not necessary if the antibodies or the agent conjugated to the antibodies do not have intracellular sites of action, e.g., if the antibody can kill the tumor cell by ADCC or some other mechanism.

The anti-Lng105 antibodies of the invention also have various non-therapeutic applications. The anti-Lng105 antibodies of the present invention can be useful for diagnosis and staging of Lng105-expressing cancers (e.g., in radioimaging). They may be used alone or in combination with other ovarian cancer markers, including, but not limited to, CA125, HE4 and mesothelin. The antibodies are also useful for purification or immunoprecipitation of Lng105 from cells, for detection and quantitation of Lng105 in vitro, e.g. in an ELISA or a Western blot, to kill and eliminate Lng105-expressing cells from a population of mixed cells as a step in the purification of other cells. The internalizing anti-Lng105 antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, immunoconjugates, and functional fragments thereof. In fusion antibodies an antibody sequence is fused to a heterologous polypeptide sequence. The antibodies can be modified in the Fc region to provide desired effector functions. As discussed in more detail in the sections below, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody competes for binding or binds substantially to, the same epitope as the antibodies of the invention. Antibodies having the biological characteristics of the present anti-Lng105 antibodies of the invention are also contemplated, e.g., an anti-Lng105 antibody which has the biological characteristics of a monoclonal antibody produced by the hybridomas accorded ATCC accession numbers PTA-5878, PTA-5879, PTA-6146, PTA-6147 and PTA-6629, specifically including the in vivo tumor targeting, internalization and any cell proliferation inhibition or cytotoxic characteristics. Specifically provided are anti-Lng105 antibodies that bind to an epitope present in amino acids 20-30, 30-40, 40-50, 50-60, 60-70, 70-80, 80-90, 90-100, 100-110, 110-120, 120-130, 130-140, 140-150, 150-160, 160-170, 170-180,180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420 of human Lng105.

Methods of producing the above antibodies are described in detail below.

The present anti-Lng105 antibodies are useful for treating an Lng105-expressing cancer or alleviating one or more symptoms of the cancer in a mammal. Such a cancer includes ovarian, pancreatic, lung or breast cancer, cancer of the urinary tract, lung cancer, breast cancer, colon cancer, pancreatic cancer, and ovarian cancer, more specifically, prostate adenocarcinoma, renal cell carcinomas, colorectal adenocarcinomas, lung adenocarcinomas, lung squamous cell carcinomas, and pleural mesothelioma. The cancers encompass metastatic cancers of any of the preceding, e.g., ovarian, pancreatic, lung or breast cancer metastases. The antibody is able to bind to at least a portion of the cancer cells that express Lng105 in the mammal and preferably is one that does not induce or that minimizes HAMA response. In a preferred embodiment, the antibody is effective to destroy or kill Lng105-expressing tumor cells or inhibit the growth of such tumor cells, in vitro or in vivo, upon binding to Lng105 on the cell. Such an antibody includes a naked anti-Lng105 antibody (not conjugated to any agent). Naked anti-Lng105 antibodies having tumor growth inhibition properties in vivo include the antibodies described in the Experimental Examples below. Naked antibodies that have cytotoxic or cell growth inhibition properties can be further harnessed with a cytotoxic agent to render them even more potent in tumor cell destruction. Cytotoxic properties can be conferred to an anti-Lng105 antibody by, e.g., conjugating the antibody with a cytotoxic agent, to form an immunoconjugate as described below. The cytotoxic agent or a growth inhibitory agent is preferably a small molecule. Toxins such as calicheamicin or a maytansinoid and analogs or derivatives thereof, are preferable.

The invention provides a composition comprising an anti-Lng105 antibody of the invention, and a carrier. For the purposes of treating cancer, compositions can be administered to the patient in need of such treatment, wherein the composition can comprise one or more anti-Lng105 antibodies present as an immunoconjugate or as the naked antibody. In a further embodiment, the compositions can comprise these antibodies in combination with other therapeutic agents such as cytotoxic or growth inhibitory agents, including chemotherapeutic agents. The invention also provides formulations comprising an anti-Lng105 antibody of the invention, and a carrier. In one embodiment, the formulation is a therapeutic formulation comprising a pharmaceutically acceptable carrier.

Another aspect of the invention is isolated nucleic acids encoding the anti-Lng105 antibodies. Nucleic acids encoding both the H and L chains and especially the hypervariable region residues, chains which encode the native sequence antibody as well as variants, modifications and humanized versions of the antibody, are encompassed.

The invention also provides methods useful for treating an Lng105-expressing cancer or alleviating one or more symptoms of the cancer in a mammal, comprising administering a therapeutically effective amount of an internalizing anti-Lng105 antibody to the mammal. The antibody therapeutic compositions can be administered short term (acute) or chronic, or intermittent as directed by physician. Also provided are methods of inhibiting the growth of, and killing an Lng105 expressing cell. Finally, the invention also provides kits and articles of manufacture comprising at least one internalizing anti-Lng105 antibody. Kits containing anti-Lng105 antibodies find use e.g., for Lng105 cell killing assays, for purification or immunoprecipitation of Lng105 from cells. For example, for isolation and purification of Lng105, the kit can contain an anti-Lng105 antibody coupled to beads (e.g., sepharose beads). Kits can be provided which contain the antibodies for detection and quantitation of Lng105 in vitro, e.g. in an ELISA or a Western blot. Such antibody useful for detection may be provided with a label such as a fluorescent or radiolabel.

Diagnostic Methods for Breast, Intestine & Colon Lung, Ovarian or Prostate Cancer

The present invention also relates to quantitative and qualitative diagnostic assays and methods for detecting, diagnosing, monitoring, staging and predicting cancers by comparing expression of Lng105 in a human patient that has or may have breast, intestine & colon, lung, ovarian or prostate cancer, or who is at risk of developing breast, intestine & colon, lung, ovarian or prostate cancer, with the expression of Lng105 in a normal human control. For purposes of the present invention, “expression of Lng105” means the amount of Lng105 that can be measured by any method known in the art.

The present invention provides methods for diagnosing breast, intestine & colon, lung, ovarian or prostate cancer in a patient, by analyzing for changes in levels of Lng105 in cells, tissues, organs or bodily fluids compared with levels of Lng105 in cells, tissues, organs or bodily fluids of preferably the same type from a normal human control, wherein an increase, or decrease in certain cases, in levels of a Lng105 in the patient versus the normal human control is associated with the presence of breast, intestine & colon, lung, ovarian or prostate cancer or with a predilection to the disease. In yet another preferred embodiment, the present invention provides methods for diagnosing breast, intestine & colon, lung, ovarian or prostate cancer in a patient by analyzing changes in a Lng105 compared to a Lng105 from a normal patient. These changes include, e.g., alterations, including post translational modifications such as glycosylation and/or phosphorylation of the Lng105 or changes in the subcellular Lng105 localization.

The present invention provides methods for diagnosing lung cancer in a patient, in particular adeno- or squamous cell carcinoma, by analyzing for changes in levels of Lng105 in cells, tissues, organs or bodily fluids compared with levels of Lng105 in cells, tissues, organs or bodily fluids of preferably the same type from a normal human control, wherein an increase, or decrease in certain cases, in levels of a Lng105 in the patient versus the normal human control is associated with the presence of lung cancer or with a predilection to the disease. In yet another preferred embodiment, the present invention provides methods for diagnosing lung cancer in a patient by analyzing changes in a Lng105 compared to a Lng105 from a normal patient. These changes include, e.g., alterations, including posttranslational modifications such as glycosylation and/or phosphorylation of the Lng105 or changes in the subcellular Lng105 localization.

For purposes of the present invention, diagnosing means that Lng105 levels are used to determine the presence or absence of disease in a patient. As will be understood by those of skill in the art, measurement of other diagnostic parameters may be required for definitive diagnosis or determination of the appropriate treatment for the disease. The determination may be made by a clinician, a doctor, a testing laboratory, or a patient using an over the counter test. The patient may have symptoms of disease or may be asymptomatic. In addition, the Lng105 levels of the present invention may be used as screening marker to determine whether further tests or biopsies are warranted. In addition, the Lng105 levels may be used to determine the vulnerability or susceptibility to disease.

In a preferred embodiment, the expression of Lng105 is measured by determining the level of Lng105, a homolog, an allelic variant, or a fragment thereof. Such levels are preferably determined in at least one of cells, tissues, organs and/or bodily fluids, including determination of normal and abnormal levels. Thus, for instance, a diagnostic assay in accordance with the invention for diagnosing over- or underexpression of Lng105 compared to normal control bodily fluids, cells, or tissue samples may be used to diagnose the presence of breast, intestine & colon, lung, ovarian or prostate cancer. The expression level of Lng105 may be determined by any method known in the art, such as those described supra. In a preferred embodiment, the Lng105 expression level may be determined by radioimmunoassays, competitive-binding assays, ELISA, Western blot, FACS, immunohistochemistry, immunoprecipitation, proteomic approaches: two-dimensional gel electrophoresis (2D electrophoresis) and non-gel-based approaches such as mass spectrometry or protein interaction profiling. See, e.g, Harlow (1999), supra; Ausubel (1992), supra; and Ausubel (1999), supra. Alterations in the Lng105 structure may be determined by any method known in the art, including, e.g., using antibodies that specifically recognize phosphoserine, phosphothreonine or phosphotyrosine residues, two-dimensional polyacrylamide gel electrophoresis (2D PAGE) and/or chemical analysis of amino acid residues of the protein. Id.

In a preferred embodiment, a radioimmunoassay (RIA) or an ELISA is used. An antibody specific to Lng105 is prepared if one is not already available. In a preferred embodiment, the antibody is a monoclonal antibody. The anti-Lng105 antibody is bound to a solid support and any free protein binding sites on the solid support are blocked with a protein such as bovine serum albumin. A sample of interest is incubated with the antibody on the solid support under conditions in which the Lng105 will bind to the anti-Lng105 antibody. The sample is removed, the solid support is washed to remove unbound material, and an anti-Lng105 antibody that is linked to a detectable reagent (a radioactive substance for RIA and an enzyme for ELISA) is added to the solid support and incubated under conditions in which binding of the Lng105 to the labeled antibody will occur. After binding, the unbound labeled antibody is removed by washing. For an ELISA, one or more substrates are added to produce a colored reaction product that is based upon the amount of an Lng105 in the sample. For an RIA, the solid support is counted for radioactive decay signals by any method known in the art. Quantitative results for both RIA and ELISA typically are obtained by reference to a standard curve.

Other methods to measure Lng105 levels are known in the art. For instance, a competition assay may be employed wherein an anti-Lng105 antibody is attached to a solid support and an allocated amount of a labeled Lng105 and a sample of interest are incubated with the solid support. The amount of labeled Lng105 attached to the solid support can be correlated to the quantity of Lng105 in the sample.

Of the proteomic approaches, 2D PAGE is a well known technique. Isolation of individual proteins from a sample such as serum is accomplished using sequential separation of proteins by isoelectric point and molecular weight. Typically, polypeptides are first separated by isoelectric point (the first dimension) and then separated by size using an electric current (the second dimension). In general, the second dimension is perpendicular to the first dimension. Because no two proteins with different sequences are identical on the basis of both size and charge, the result of 2D PAGE is a roughly square gel in which each protein occupies a unique spot. Analysis of the spots with chemical or antibody probes, or subsequent protein microsequencing can reveal the relative abundance of a given protein and the identity of the proteins in the sample.

The above tests can be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. As used herein “blood” includes whole blood, plasma, serum, circulating epithelial cells, constituents, or any derivative of blood.

In addition to detection in bodily fluids, the proteins of the invention are suitable to detection by cell capture technology. Whole cells may be captured by a variety methods for example magnetic separation, U.S. Pat. Nos. 5,200,084; 5,186,827; 5,108,933; 4,925,788, the disclosures of which are incorporated herein by reference in their entireties. Epithelial cells may be captured using such products as Dynabeads®) or CELLection™ (Dynal Biotech, Oslo, Norway). Alternatively, fractions of blood may be captured, e.g., the buffy coat fraction (50 mm cells isolated from 5 ml of blood) containing epithelial cells. In addition, cancer cells may be captured using the techniques described in WO 00/47998, the disclosure of which is incorporated herein by reference in its entirety. Once the cells are captured or concentrated, the proteins or nucleic acids are detected by the means described in the subject application. Alternatively, nucleic acids may be captured directly from blood samples, see U.S. Pat. Nos. 6,156,504, 5,501,963; or WO 01/42504, the disclosures of which are incorporated herein by reference in their entireties.

In a preferred embodiment, the specimen tested for expression of Lng105 includes without limitation normal or cancerous breast, intestine & colon, lung, ovarian or prostate tissue, normal or cancerous breast, intestine & colon, lung, ovarian or prostate cells grown in cell culture, blood, serum, lymph node tissue, and lymphatic fluid. In another preferred embodiment, especially when metastasis of a primary breast, intestine & colon, lung, ovarian or prostate cancer is known or suspected, specimens include, without limitation, tissues from brain, bone, bone marrow, liver, lungs, colon, and adrenal glands. In general, the tissues may be sampled by biopsy, including, without limitation, needle biopsy, e.g., transthoracic needle aspiration, cervical mediatinoscopy, endoscopic lymph node biopsy, video-assisted thoracoscopy, exploratory thoracotomy, bone marrow biopsy and bone marrow aspiration.

All the methods of the present invention may optionally include determining the expression levels of one or more other cancer markers in addition to determining the expression level of Lng105. In many cases, the use of another cancer marker will decrease the likelihood of false positives or false negatives. Other cancer markers useful in the present invention will depend on the cancer being tested and are known to those of skill in the art. In a preferred embodiment, at least one other cancer marker in addition to a particular Lng105 is measured. In a more preferred embodiment, at least two other additional cancer markers are used. In an even more preferred embodiment, at least three, more preferably at least five, even more preferably at least ten additional cancer markers are used.

In a preferred embodiment, the specimen tested for expression of Lng105 includes without limitation colon tissue, fecal samples, colonocytes, colon cells grown in cell culture, blood, serum, lymph node tissue, and lymphatic fluid. In another preferred embodiment, especially when metastasis of a primary colon cancer is known or suspected, specimens include, without limitation, tissues from brain, bone, bone marrow, liver, lungs, and adrenal glands. In general, the tissues may be sampled by biopsy, including, without limitation, needle biopsy, e.g., transthoracic needle aspiration, cervical mediatinoscopy, endoscopic lymph node biopsy, video-assisted thoracoscopy, exploratory thoracotomy, bone marrow biopsy and bone marrow aspiration.

Colonocytes represent an important source of the Lng105 because they provide a picture of the immediate past metabolic history of the GI tract of a subject. In addition, such cells are representative of the cell population from a statistically large sampling frame reflecting the state of the colonic mucosa along the entire length of the colon in a non-invasive manner, in contrast to a limited sampling by colonic biopsy using an invasive procedure involving endoscopy. Specific examples of patents describing the isolatation colonocytes include U.S. Pat. Nos. 6,335,193; 6,020,137 5,741,650; 6,258,541; US 2001 0026925 A1; WO 00/63358 A1, the disclosures of which are incorporated herein by reference in their entireties.

In a preferred embodiment, the specimen tested for expression of Lng105 includes, without limitation, Lung tissue, fluid obtained by bronchial alveolar lavage (BAL), sputum, Lung cells grown in cell culture, blood, serum, lymph node tissue and lymphatic fluid. In another preferred embodiment, especially when metastasis of a primary Lung cancer is known or suspected, specimens include, without limitation, tissues from brain, bone, bone marrow, liver, adrenal glands and colon. In general, the tissues may be sampled by biopsy, including, without limitation, needle biopsy, e.g., transthoracic needle aspiration, cervical mediatinoscopy, endoscopic lymph node biopsy, video-assisted thoracoscopy, exploratory thoracotomy, bone marrow biopsy and bone marrow aspiration. See Scott, supra and Franklin, pp. 529-570, in Kane, supra. For early and inexpensive detection, assaying for changes in Lng105 in cells in sputum samples may be particularly useful. Methods of obtaining and analyzing sputum samples are disclosed in Franklin, supra.

All the methods of the present invention may optionally include determining the expression levels of one or more other cancer markers in addition to determining the expression level Lng105. In many cases, the use of another cancer marker will decrease the likelihood of false positives or false negatives. Other cancer markers useful in the present invention will depend on the cancer being tested and are known to those of skill in the art. In a preferred embodiment, at least one other cancer marker in addition to a particular Lng105 is measured. In a more preferred embodiment, at least two other additional cancer markers are used. In an even more preferred embodiment, at least three, more preferably at least five, even more preferably at least ten additional cancer markers are used.

For prostate cancer, the progress of therapy can be assessed by routine methods, usually by measuring serum PSA (prostate specific antigen) levels; the higher the level of PSA in the blood, the more extensive the cancer.

Commercial assays for detecting PSA are available, e.g, Hybitech Tandem-E and Tandem-R PSA assay kits, the Yang ProsCheck polyclonal assay (Yang Labs, Bellevue, Wash.), Abbott Inx (Abbott Labs, Abbott Park, Ill.), etc. Metastasis can be determined by staging tests and by bone scan and tests for calcium level and other enzymes to determine spread to the bone, CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

For bladder cancer, which is a more localized cancer, methods to determine progress of disease include urinary cytologic evaluation by cystoscopy, monitoring for presence of blood in the urine, visualization of the urothelial tract by sonography or an intravenous pyelogram, computed tomography (CT) and magnetic resonance imaging (MRI). The presence of distant metastases can be assessed by CT of the abdomen, chest x-rays, or radionuclide imaging of the skeleton.

Diagnosing

In one aspect, the invention provides a method for determining the expression levels and/or structural alterations of Lng105 in a sample from a patient suspected of having breast, intestine & colon, lung, ovarian or prostate cancer. In general, the method comprises the steps of obtaining the sample from the patient, determining the expression level or structural alterations of Lng105 and then ascertaining whether the patient has breast, intestine & colon, lung, ovarian or prostate cancer from the expression level of Lng105. In general, if high expression relative to a control of a Lng105 is indicative of breast, intestine & colon, lung, ovarian or prostate cancer, a diagnostic assay is considered positive if the level of expression of the Lng105 is at least one and a half times higher, and more preferably are at least two times higher, still more preferably five times higher, even more preferably at least ten times higher, than in preferably the same cells, tissues or bodily fluid of a normal human control. In contrast, if low expression relative to a control of a Lng105 is indicative of breast, intestine & colon, lung, ovarian or prostate cancer, a diagnostic assay is considered positive if the level of expression of the Lng105 is at least one and a half times lower, and more preferably are at least two times lower, still more preferably five times lower, even more preferably at least ten times lower than in preferably the same cells, tissues or bodily fluid of a normal human control. The normal human control may be from a different patient or from uninvolved tissue of the same patient.

The present invention also provides a method of determining whether breast, intestine & colon, lung, ovarian or prostate cancer has metastasized in a patient. One may identify whether the breast, intestine & colon, lung, ovarian or prostate cancer has metastasized by measuring the expression levels and/or structural alterations of Lng105 in a variety of tissues. The presence of Lng105 in a certain tissue at levels higher than that of corresponding noncancerous tissue (e.g., the same tissue from another individual) is indicative of metastasis if high level expression of a Lng105 is associated with breast, intestine & colon, lung, ovarian or prostate cancer. Similarly, the presence of a Lng105 in a tissue at levels lower than that of corresponding noncancerous tissue is indicative of metastasis if low level expression of a Lng105 is associated with breast, intestine & colon, lung, ovarian or prostate cancer. Further, the presence of a structurally altered Lng105 that is associated with breast, intestine & colon, lung, ovarian or prostate cancer is also indicative of metastasis.

In general, if high expression relative to a control of a Lng105 is indicative of metastasis, an assay for metastasis is considered positive if the level of expression of the Lng105 is at least one and a half times higher, and more preferably are at least two times higher, still more preferably five times higher, even more preferably at least ten times higher, than in preferably the same cells, tissues or bodily fluid of a normal human control. In contrast, if low expression relative to a control of a Lng105 is indicative of metastasis, an assay for metastasis is considered positive if the level of expression of the Lng105 is at least one and a half times lower, and more preferably are at least two times lower, still more preferably five times lower, even more preferably at least ten times lower than in preferably the same cells, tissues or bodily fluid of a normal human control.

Staging

The invention also provides a method of staging breast, intestine & colon, lung, ovarian or prostate cancer in a human patient. The method comprises identifying a human patient having breast, intestine & colon, lung, ovarian or prostate cancer and analyzing cells, tissues or bodily fluids from such human patient for expression levels and/or structural alterations of Lng105. First, one or more tumors from a variety of patients are staged according to procedures well known in the art, and the expression levels of Lng105 is determined for each stage to obtain a standard expression level for Lng105. Then, the Lng105 expression levels of the Lng105 are determined in a biological sample from a patient whose stage of cancer is not known. The Lng105 expression levels from the patient are then compared to the standard expression level. By comparing the expression level of the Lng105 from the patient to the standard expression levels, one may determine the stage of the tumor. The same procedure may be followed using structural alterations of a Lng105 to determine the stage of a breast, intestine & colon, lung, ovarian or prostate cancer.

Monitoring

Further provided is a method of monitoring breast, intestine & colon, lung, ovarian or prostate cancer in a human patient. One may monitor a human patient to determine whether there has been metastasis and, if there has been, when metastasis began to occur. One may also monitor a human patient to determine whether a preneoplastic lesion has become cancerous. One may also monitor a human patient to determine whether a therapy, e.g., chemotherapy, radiotherapy or surgery, has decreased or eliminated the breast, intestine & colon, lung, ovarian or prostate cancer. The monitoring may determine if there has been a reoccurrence and, if so, determine its nature. The method comprises identifying a human patient that one wants to monitor for breast, intestine & colon, lung, ovarian or prostate cancer, periodically analyzing cells, tissues or bodily fluids from such human patient for expression levels of Lng105, and comparing the Lng105 levels over time to those Lng105 expression levels obtained previously. Patients may also be monitored by measuring one or more structural alterations in a Lng105 that are associated with breast, intestine & colon, lung, ovarian or prostate cancer.

If increased expression of a Lng105 is associated with metastasis, treatment failure, or conversion of a preneoplastic lesion to a cancerous lesion, then detecting an increase in the expression level of a Lng105 indicates that the tumor is metastasizing, that treatment has failed or that the lesion is cancerous, respectively. One having ordinary skill in the art would recognize that if this were the case, then a decreased expression level would be indicative of no metastasis, effective therapy or failure to progress to a neoplastic lesion. If decreased expression of a Lng105 is associated with metastasis, treatment failure, or conversion of a preneoplastic lesion to a cancerous lesion, then detecting a decrease in the expression level of a Lng105 indicates that the tumor is metastasizing, that treatment has failed or that the lesion is cancerous, respectively. In a preferred embodiment, the levels of Lng105 are determined from the same cell type, tissue or bodily fluid as prior patient samples. Monitoring a patient for onset of breast, intestine & colon, lung, ovarian or prostate cancer metastasis is periodic and preferably is done on a quarterly basis, but may be done more or less frequently.

The methods described herein can farther be utilized as prognostic assays to identify subjects having or at risk of developing a disease or disorder associated with increased or decreased expression levels of a Lng105. The present invention provides a method in which a test sample is obtained from a human patient and one or more Lng105 are detected. The presence of higher (or lower) Lng105 levels as compared to normal human controls is diagnostic for the human patient being at risk for developing cancer, particularly breast, intestine & colon, lung, ovarian or prostate cancer. The effectiveness of therapeutic agents to decrease (or increase) expression or activity of one or more Lng105 of the invention can also be monitored by analyzing levels of expression of the Lng105 in a human patient in clinical trials or in in vitro screening assays such as in human cells. In this way, the gene expression pattern can serve as a marker, indicative of the physiological response of the human patient or cells, as the case may be, to the agent being tested.

Methods of Detecting Noncancerous Breast, Intestine & Colon, Lung, Ovarian or Prostate Diseases

The present invention also provides methods for determining the expression levels and/or structural alterations of one or more Lng105 in a sample from a patient suspected of having or known to have a noncancerous breast, intestine & colon, lung, ovarian or prostate disease. In general, the method comprises the steps of obtaining a sample from the patient, determining the expression level or structural alterations of a Lng105, comparing the expression level or structural alteration of the Lng105 to a normal breast, intestine & colon, lung, ovarian or prostate control, and then ascertaining whether the patient has a noncancerous breast, intestine & colon, lung, ovarian or prostate disease. In general, if high expression relative to a control of a Lng105 is indicative of a particular noncancerous breast, intestine & colon, lung, ovarian or prostate disease, a diagnostic assay is considered positive if the level of expression of the Lng105 is at least two times higher, and more preferably are at least five times higher, even more preferably at least ten times higher, than in preferably the same cells, tissues or bodily fluid of a normal human control. In contrast, if low expression relative to a control of a Lng105 is indicative of a noncancerous breast, intestine & colon, lung, ovarian or prostate disease, a diagnostic assay is considered positive if the level of expression of the Lng105 is at least two times lower, more preferably are at least five times lower, even more preferably at least ten times lower than in preferably the same cells, tissues or bodily fluid of a normal human control. The normal human control may be from a different patient or from uninvolved tissue of the same patient.

One having ordinary skill in the art may determine whether a Lng105 is associated with a particular noncancerous breast, intestine & colon, lung, ovarian or prostate disease by obtaining breast, intestine & colon, lung, ovarian or prostate tissue from a patient having a noncancerous breast, intestine & colon, lung, ovarian or prostate disease of interest and determining if Lng105 are expressed in the tissue at either a higher or a lower level than in normal breast, intestine & colon, lung, ovarian or prostate tissue. In another embodiment, one may determine whether a Lng105 exhibits structural alterations in a particular noncancerous breast, intestine & colon, lung, ovarian or prostate disease state by obtaining breast, intestine & colon, lung, ovarian or prostate tissue from a patient having a noncancerous breast, intestine & colon, lung, ovarian or prostate disease of interest and determining the structural alterations in Lng105 relative to normal breast, intestine & colon, lung, ovarian or prostate tissue.

Methods for Identifying Breast, Intestine & Colon, Lung, Ovarian or Prostate Tissue

In another aspect, the invention provides methods for identifying breast, intestine & colon, lung, ovarian or prostate tissue. These methods are particularly useful in, e.g., forensic science, breast, intestine & colon, lung, ovarian or prostate cell differentiation and development, and in tissue engineering. In one embodiment, the invention provides a method for determining whether a sample is breast, intestine & colon, lung, ovarian or prostate tissue or has breast, intestine & colon, lung, ovarian or prostate tissue-like characteristics. The method comprises the steps of providing a sample suspected of comprising breast, intestine & colon, lung, ovarian or prostate tissue or having breast, intestine & colon, lung, ovarian or prostate tissue-like characteristics, determining whether the sample expresses Lng105, and, if the sample expresses Lng105, concluding that the sample comprises breast, intestine & colon, lung, ovarian or prostate tissue. In another preferred embodiment, the method can be practiced by determining whether Lng105 is expressed. Determining whether a sample expresses Lng105 can be accomplished by any method known in the art. Preferred methods include Western blot, ELISA, RIA and 2D PAGE. one embodiment is Lng105 or a homolog, allelic variant or fragment thereof.

In one embodiment, the method can be used to determine whether an unknown tissue is breast, intestine & colon, lung, ovarian or prostate tissue. This is particularly useful in forensic science, in which small, damaged pieces of tissues that are not identifiable by microscopic or other means are recovered from a crime or accident scene. In another embodiment, the method can be used to determine whether a tissue is differentiating or developing into breast, intestine & colon, lung, ovarian or prostate tissue. This is important in monitoring the effects of the addition of various agents to cell or tissue culture, e.g., in producing new breast, intestine & colon, lung, ovarian or prostate tissue by tissue engineering. These agents include, e.g., growth and differentiation factors, extracellular matrix proteins and culture medium. Other factors that may be measured for effects on tissue development and differentiation include gene transfer into the cells or tissues, alterations in pH, aqueous:air interface and various other culture conditions.

Production of Anti-Lng105 Antibodies

The following describes exemplary techniques for the production of the antibodies useful in the present invention. Some of these techniques are described further in Example 1. The Lng105 antigen to be used for production of antibodies may be, e.g., the fall length polypeptide or a portion thereof, including a soluble form of Lng105 lacking the membrane spanning sequence, or synthetic peptides to selected portions of the protein.

Alternatively, cells expressing Lng105.At their cell surface (e.g. CHO or NIH-3T3 cells transformed to overexpress Lng105; ovarian, pancreatic, lung or breast or other Lng105-expressing tumor cell line), or membranes prepared from such cells can be used to generate antibodies. The nucleotide and amino acid sequences of human and murine Lng105.Are available as provided above. Lng105 can be produced recombinantly in and isolated from, bacterial or eukaryotic cells using standard recombinant DNA methodology. Lng105 can be expressed as a tagged (e.g., epitope tag) or other fusion protein to facilitate isolation as well as identification in various assays.

Antibodies or binding proteins that bind to various tags and fusion sequences are available as elaborated below. Other forms of Lng105 useful for generating antibodies will be apparent to those skilled in the art.

Tags

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. The FLAG-peptide [Hopp et al., BioTechnology, 6:1204-1210 (1988)] is recognized by an anti-FLAG M2 monoclonal antibody (Eastman Kodak Co., New Haven, Conn.). Purification of a protein containing the FLAG peptide can be performed by immunoaffinity chromatography using an affinity matrix comprising the anti-FLAG M2 monoclonal antibody covalently attached to agarose (Eastman Kodak Co., New Haven, Conn.). Other tag polypeptides include the KT3 epitope peptide [Martin et al., Science, 255:192-194 (1992)]; an α-tubulin epitope peptide [Skinner et al., J. Biol. Chenz., 266:15163-15166 (1991)]; and the T7 gene protein peptide tag [Lutz-Freyermuth et al., Proc. Natl. Acad. Sci. USA, 87:6393-6397 (1990)].

Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulf6succinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 pg or 5 pg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies. Principles and Practice, pp 103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-II mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980). Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp 103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Biotechnology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and CL) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide (heterologous polypeptide). The nonimmunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Humanized Antibodies

Methods for humanizing non-human antibodies have been described in the art. Preferably, a humanized antibody has one or more amino acid residues introduced into it from a source which is nonhuman. These non-human amino acid residues are often referred to as “import” residues, which are typically taken from an “import” variable domain. Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art.

Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-Lng105 antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

Human Antibodies

As an alternative to humanization, human antibodies can be generated. For example, it is now possible to produce transgenic animals (e.g., mice) that are capable, upon immunization, of producing a fall repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669 (all of GenPharm); 5,545,807; and Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. As discussed above, human antibodies may also be generated by in vitro activated B cells (see U.S. Pat. Nos. 5,567,610 and 5,229,275).

Antibody Fragments

In certain circumstances there are advantages of using antibody fragments, rather than whole antibodies. The smaller size of the fragments allows for rapid clearance, and may lead to improved access to solid tumors. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab)2 fragments (Carter et al., Bio/Technology 10: 163-167 (1992)). According to another approach, F(ab)2 fragments can be isolated directly from recombinant host cell culture. Fab and F(ab)2 fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Bispecific Antibodies

Bispecific antibodies are antibodies that have binding specificities for at least two different epitopes. Exemplary bispecific antibodies may bind to two different epitopes of the Lng105 protein. Other such antibodies may combine an Lng105 binding site with a binding site for another protein. Alternatively, an anti-Lng105.Arm may be combined with an arm which binds to a triggering molecule on a leukocyte such as a Tcell receptor molecule (e.g. C133), or Fc receptors for IgG (FcγR), such as FcγRI (CD64), FcγRlI (CD32) and FcγRIII (CD16), so as to focus and localize cellular defense mechanisms to the Lng105-expressing cell. Bispecific antibodies may also be used to localize cytotoxic agents to cells which express Lng105. These antibodies possess an Lng105-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab)2 bispecific antibodies). WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J, 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, CH², and CH3 regions. It is preferred to have the first heavy-chain constant region (CHI) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

The “diabody” technology described by Hollinger et al., Proc. Natl. Acad. Sci. USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1(X1n-VD2-(X2)n-Fc, wherein VDI is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CHI-flexible linker-VH-CHI-Fc region chain; or VHCHI-VH-CHI-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Other Amino Acid Sequence Modifications

Amino acid sequence modification(s) of the anti-Lng105 antibodies described herein are contemplated. For example, it may be desirable to improve the binding affinity and/or other biological properties of the antibody. Amino acid sequence variants of the anti-Lng105 antibody are prepared by introducing appropriate nucleotide changes into the anti-Lng105 antibody nucleic acid, or by peptide synthesis.

Such modifications include, for example, deletions from, and/or insertions into and/or substitutions of, residues within the amino acid sequences of the anti-Lng105 antibody. Any combination of deletion, insertion, and substitution is made to arrive at the final construct, provided that the final construct possesses the desired characteristics. The amino acid changes also may alter post-translational processes of the anti-Lng105 antibody, such as changing the number or position of glycosylation sites.

A useful method for identification of certain residues or regions of the anti-Lng105 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with Lng105 antigen.

Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-Lng105 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-Lng105 antibody with an N-terminal methionyl residue or the antibody fused to a cytotoxic polypeptide. Other insertional variants of the anti-Lng105 antibody molecule include the fusion to the N- or C-terminus of the anti-Lng105 antibody to an enzyme (e.g. for ADEPT) or a polypeptide which increases the serum half-life of the antibody.

Another type of variant is an amino acid substitution variant. These variants have at least one amino acid residue in the anti-Lng105 antibody molecule replaced by a different residue. The sites of greatest interest for substitutional mutagenesis include the hypervariable regions, but FR alterations are also contemplated. Conservative substitutions are shown in Table I under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE I
Amino Acid Substitutions

| Original | Exemplary Substitutions | Preferred Substitutions |
|---|---|---|
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties.

(1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the anti-Lng105 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human Lng105. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-Lng105 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-Lng105 antibody.

It may be desirable to modify the antibody of the invention with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of the antibody.

Screening for Antibodies with the Desired Properties

Techniques for generating antibodies have been described above. One may further select antibodies with certain biological characteristics, as desired.

The growth inhibitory effects of an anti-Lng105 antibody of the invention may be assessed by methods known in the art, e.g., using cells which express Lng105 either endogenously or following transfection with the Lng105 gene. For example, the tumor cell lines and Lng105-transfected cells provided in Example 1 below may treated with an anti-Lng105 monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-Lng105 antibody of the invention. After antibody treatment, the cells are harvested and the amount of radioactivity incorporated into the DNA quantitated in a scintillation counter. Appropriated positive controls include treatment of a selected cell line with a growth inhibitory antibody known to inhibit growth of that cell line. Growth inhibition of tumor cells in vivo can be determined in various ways such as is described in the Experimental Examples section below. Preferably, the tumor cell is one that over-expresses Lng105. Preferably, the anti-Lng105 antibody will inhibit cell proliferation of an Lng105-expressing tumor cell in vitro or in vivo by about 25-100% compared to the untreated tumor cell, more preferably, by about 30-100%, and even more preferably by about 50-100% or 70-100%, at an antibody concentration of about 0.5 to 30 μg/ml. Growth inhibition can be measured at an antibody concentration of about 0.5 to 30 μg/ml or about 0.5 nM to 200 nM in cell culture, where the growth inhibition is determined 1-10 days after exposure of the tumor cells to the antibody. The antibody is growth inhibitory in vivo if administration of the anti-Lng105 antibody at about 1 μg/kg to about 100 mg/kg body weight results in reduction in tumor size or tumor cell proliferation within about 5 days to 3 months from the first administration of the antibody, preferably within about 5 to 30 days.

To select for antibodies which induce cell death, loss of membrane integrity as indicated by, e.g., propidium iodide (PI), trypan blue or 7AAD uptake may be assessed relative to control. A PI uptake assay can be performed in the absence of complement and immune effector cells. Lng105-expressing tumor cells are incubated with medium alone or medium containing of the appropriate monoclonal antibody at e.g., about 10 μg/ml. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN™ flow cytometer and FACSCONVERT™ CellQuest software (Becton Dickinson). Those antibodies which induce statistically significant levels of cell death as determined by PI uptake may be selected as cell death-inducing antibodies.

To screen for antibodies which bind to an epitope on Lng105 bound by an antibody of interest, a routine cross-blocking assay such as that describe in Antibodies, A Laboratory Manual, Cold Spring Harbor Laboratory, Ed Harlow and David Lane (1988), can be performed. This assay can be used to determine if a test antibody binds the same site or epitope as an anti-Lng105 antibody of the invention. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of Lng105 can be used in competition assays with the test antibodies or with a test antibody and an antibody with a characterized or known epitope.

Immunoconjugates

The invention also pertains to therapy with immunoconjugates comprising an antibody conjugated to an anti-cancer agent such as a cytotoxic agent or a growth inhibitory agent.

Chemotherapeutic agents useful in the generation of such immunoconjugates have been described above. Conjugates of an antibody and one or more small molecule toxins, such as a calicheamicin, maytansinoids, a trichothene, and CC1065, and the derivatives of these toxins that have toxin activity, are also contemplated herein.

Maytansine and Maytansinoids

In one preferred embodiment, an anti-Lng105 antibody (full length or fragments) of the invention is conjugated to one or more maytansinoid molecules. Maytansinoids are mitotic inhibitors which act by inhibiting tubulin polymerization. Maytansine was first isolated from the cast African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansinoid-Antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DMI linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1 -maytansonoid conjugate was tested in vitro on the human breast cancer cell line SK-BR-3, which expresses 3×105 HER-2 surface antigens per cell. The drug conjugate achieved a degree of cytotoxicity similar to the free maytansonoid drug, which could be increased by increasing the number of maytansinoid molecules per antibody molecule. The A7-maytansinoid conjugate showed low systemic cytotoxicity in mice.

Anti-Lng105 Antibody-Maytansinoid Conjugates (Immunoconjugates)

Anti-Lng105 antibody-maytansinoid conjugates are prepared by chemically linking an anti-Lng105 antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al. Cancer Research 52: 127-131 (1992). The linking groups include disulfide groups, thioether groups, acid labile groups, photolabile groups, peptidase labile groups, or esterase labile groups, as disclosed in the above identified patents, disulfide and thioether groups being preferred. Conjugates of the antibody and maytansinoid may be made using a variety of bifunctional protein coupling agents such as N-succinimidyl (2-pyridyidithio) propionate (SPDP), succinimidyl- (N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as his (p-azidobelizoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). Particularly preferred coupling agents include N-succinimidyl (2-pyridyldithio) propionate (SPDP) (Carlsson et al., Biochem. J. 173:723-737 [1978]) and N-succinimidyl (2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an anti-Lng105 antibody conjugated to one or more calicheamicin molecules. The calicheamicin family of antibiotics is capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, and 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ₁^{I}, (Hinman et al. Cancer Research 53: 3336 (1993), Lode et al. Cancer Research 5 8: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents

Other antitumor agents that can be conjugated to the anti-Lng105 antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. NO. 5,877,296). Enzymatically active toxins and fragments thereof which can be used include diphtheria A chain, 1 5 nonbinding active fragments of diphtheria toxin, exotoxin A chain (from Pseudomoinas aerugiizosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993. The present invention further contemplates an immunoconjugate formed between an antibody and a compound with nucleolytic activity (e.g. a ribonuclease or a DNA endonuclease such as a deoxyribonuclease; DNase).

For selective destruction of the tumor, the antibody may comprise a highly radioactive atom. A variety of radioactive isotopes are available for the production of radioconjugated anti-Lng105 antibodies. Examples include At²¹¹, I¹³¹, I¹²⁵, In¹¹¹, Y⁹⁰, Re¹⁸⁶, Re¹⁸⁸, Sm¹⁵³, Bi²¹², P³², and radioactive isotopes of Lu. When the conjugate is used for diagnosis, it may comprise a radioactive atom for scintigraphic studies, for example tc^{99M} or I¹²³, or a spin label for nuclear magnetic resonance (NMR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99M}, I¹²³, In¹¹¹, Re¹⁸⁶, Re¹⁸⁸, can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl (2-pyridyldithio) propionate (SPDP), succinimidyl (N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon labeled 1-isothiocyanatobenzyl methyldiethylene triaminepentaacetic acid (MX-DTPA) is an exemplary chelating agent for conjugation of radionucleotide to the antibody. See WO 94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

Alternatively, a fusion protein comprising the anti-Lng105 antibody and cytotoxic agent may be made, e.g. by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

Antibody Dependent Enzyme Mediated Prodrug Therapy (ADEPT)

The antibodies of the present invention may also be used in ADEPT by conjugating the antibody to a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anti-cancer drug, 5-fluorouracil; proteases, such as serratia protease, thermolysin, subtili sin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuramrinidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population. The enzymes of this invention can be covalently bound to the anti-Lng105 antibodies by techniques well known in the art such as the use of the heterobifunctional crosslinking reagents discussed above.

Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The anti-Lng105 antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA, 82:3688 (1985); Hwang et al., Proc. Natl Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst.81(19)1484 (1989).

Vectors, Host Cells, and Recombinant Methods

The invention also provides isolated nucleic acid encoding the humanized anti-Lng105 antibody, vectors and host cells comprising the nucleic acid, and recombinant techniques for the production of the antibody. For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

Signal Sequence Component

The anti-Lng105 antibody of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native anti-Lng105 antibody signal sequence, the signal sequence is substituted by a prokaryotic signal sequence selected, for example, from the group of the alkaline phosphatase, penicillinase, 1 pp, or heat-stable enterotoxin II leaders. For yeast secretion the native signal sequence may be substituted by, e.g., the yeast invertase leader, oc factor leader (including Saccharomyces and Kluyveromyces cc-factor leaders), or acid phosphatase leader, the C albicans glucoamylase leader, or the signal described in WO 90/13646. In mammalian cell expression, mammalian signal sequences as well as viral secretory leaders, for example, the herpes simplex gD signal, are available. The DNA for such precursor region is ligated in reading frame to DNA encoding the anti-Lng105 antibody.

Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the anti-Lng105 antibody nucleic acid, such as DHFR, thymidine kinase, metallothionein-I and -11, preferably primate metallothionein genes, adenosine deaminase, omithine decarboxylase, etc. For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding anti-Lng105 antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4 Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 pm circular plasmid pKDI can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Promoter Component

Expression and cloning vectors usually contain a promoter that is recognized by the host organism and is operably linked to the anti-Lng105 antibody nucleic acid. Promoters suitable for use with prokaryotic hosts include the phoA promoter, P-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the anti-Lng105 antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors. Examples of suitable promoter sequences for use with yeast hosts include the promoters for 3-phosphoglycerate kinase or other glycolytic enzymes, such as enolase, glyceraldehyde phosphate dehydrogenase, hexokinase, pyruvate decarboxylase, phosphofructokinase, glucose phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde phosphate dehydrogenase, and enzymes responsible for maltose and galactose utilization. Suitable vectors and promoters for use in yeast expression are further described in EP 73,657. Yeast enhancers also are advantageously used with yeast promoters.

Anti-Lng105 antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIll E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human P-interferon cDNA in mouse cells under the control of a thymidine kinase promoter from herpes simplex virus. Alternatively, the Rous Sarcoma Virus long terminal repeat can be used as the promoter.

Enhancer Element Component

Transcription of a DNA encoding the anti-Lng105 antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the anti-Lng105 antibody-encoding sequence, but is preferably located at a site 5′ from the promoter.

Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′ untranslated regions of eukaryotic or viral DNAs or cDNAs. These regions contain nucleotide segments transcribed as polyadenylated fragments in the untranslated portion of the MRNA encoding anti-Lng105 antibody. One useful transcription termination component is the bovine growth hormone polyadenylation region. See WO 94/11026 and the expression vector disclosed therein.

Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W31 10 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for anti-Lng105 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwalniiomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-Lng105 antibody are derived from multicellular organisms. Examples of invertebrate cells include plant and insect cells. Numerous baculoviral strains and variants and corresponding permissive insect host cells from hosts such as Spodopterafrugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980) ); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, 1413 8065); mouse mammary tumor (MMT 060562, ATCC CCL5 1); TRI cells (Mather et al., Annals N. Y Acad. Sci. 383:44-68 (1982)); MRC 5 cells; FS4 cells; and a human hepatoma line (Hep G2).

Host cells are transformed with the above-described expression or cloning vectors for anti-Lng105 antibody production and cultured in conventional nutrient media modified as appropriate for inducing promoters, selecting transformants, or amplifying the genes encoding the desired sequences.

Culturing Host Cells

The host cells used to produce the anti-Lng105 antibody of this invention may be cultured in a variety y of media. Commercially available media such as Ham's FIO (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem.102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Purification of anti-Lng105 Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SIDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

V. Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol, and mcresol); low molecular weight (less than about 10 residues) polypeptides; proteins, such as serum albumin, gelatin, or immunoglobulins; hydrophilic polymers such as polyvinylpyllolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g. Zn-protein complexes); and/or non-ionic surfactants such as TWEEN™, PLURONICS™ or polyethylene glycol (PEG). The antibody preferably comprises the antibody at a concentration of between 5-200 mg/ml, preferably between 10-100 mg/ml.

The formulation herein may also contain more than one active compound as necessary for the particular indication being treated, preferably those with complementary activities that do not adversely affect each other. For example, in addition to the anti-Lng105 antibody which internalizes, it may be desirable to include in the one formulation, an additional antibody, e.g. a second anti-Lng105 antibody which binds a different epitope on Lng105, or an antibody to some other target such as a growth factor that affects the growth of the particular cancer. Alternatively, or additionally, the composition may farther comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-Lglutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−) hydroxybutyric acid.

The formulations to be used for in vivo administration must be sterile. This is readily accomplished by filtration through sterile filtration membranes.

Treatment Using Anti-Lng105 Antibodies

According to the present invention, the anti-Lng105 antibody that internalizes upon binding Lng105 on a cell surface is used to treat an Lng105-expressing cancer cell, in particular, ovarian, pancreatic, lung or breast cancer, such as ovarian serous adenocarcinoma or breast infiltrating ductal carcinoma cancer, and associated metastases.

The cancer will generally comprise Lng105-expressing cells, such that the anti-Lng105 antibody is able to bind thereto. While the cancer may be characterized by overexpression of the Lng105 molecule, the present application further provides a method for treating cancer which is not considered to be an Lng105-overexpressing cancer.

To determine Lng105 expression in the cancer, various diagnostic assays are available. In one embodiment, Lng105 overexpression may be analyzed by immunohistochemistry (IHC). Parrafin embedded tissue sections from a tumor biopsy may be subjected to the IHC assay and accorded an Lng105 protein staining intensity criteria as follows.

Score 0 no staining is observed or membrane staining is observed in less than 10% of tumor cells.

Score 1+ a faint/barely perceptible membrane staining is detected in more than 10% of the tumor cells. The cells are only stained in part of their membrane.
Score 2+ a weak to moderate complete membrane staining is observed in more than 10% of the tumor cells.

Score 3+ a moderate to strong complete membrane staining is observed in more than 10% of the tumor cells.

Those tumors with 0 or 1+ scores for Lng105 expression may be characterized as not overexpressing Lng105, whereas those tumors with 2+ or 3+ scores may be characterized as overexpressing Lng105.

Alternatively, or additionally, FISH assays such as the INFORM™ (sold by Ventana, Ariz.) or PATHVISION™ (VySiS, Ill.) may be carried out on formalin-fixed, paraffin-embedded tumor tissue to determine the extent (if any) of Lng105 overexpression in the tumor. Lng105 overexpression or amplification may be evaluated using an in vivo diagnostic assay, e.g. by administering a molecule (such as an antibody) which binds the molecule to be detected and is tagged with a detectable label (e.g. a radioactive isotope or a fluorescent label) and externally scanning the patient for localization of the label.

Currently, depending on the stage of the cancer, ovarian, pancreatic, lung or breast cancer treatment involves one or a combination of the following therapies: surgery to remove the cancerous tissue, radiation therapy, androgen deprivation (e.g., hormonal therapy), and chemotherapy. Anti-Lng105 antibody therapy may be especially desirable in elderly patients who do not tolerate the toxicity and side effects of chemotherapy well, in metastatic disease where radiation therapy has limited usefulness, and for the management of prostatic carcinoma that is resistant to androgen deprivation treatment. The tumor targeting and internalizing anti-Lng105 antibodies of the invention are useful to alleviate Lng105-expressing cancers, e.g., lung upon initial diagnosis of the disease or during relapse. For therapeutic applications, the anti-Lng105 antibody can be used alone, or in combination therapy with, e.g., hormones, antiangiogens, or radiolabelled compounds, or with surgery, cryotherapy, and/or radiotherapy, notably for lung, also particularly where shed cells cannot be reached. Anti-Lng105 antibody treatment can be administered in conjunction with other forms of conventional therapy, either consecutively with, pre- or post-conventional therapy, Chemotherapeutic drugs such as taxotere® (docetaxel), taxol® (paclitaxel), estramustine and mitoxantrone are used in treating metastatic and hormone refractory ovarian, pancreatic, lung or breast cancer, in particular, in good risk patients. In the present method of the invention for treating or alleviating cancer, in particular, androgen independent and/or metastatic ovarian, pancreatic, lung or breast cancer, the cancer patient can be administered anti-Lng105 antibody in conjunction with treatment with the one or more of the preceding chemotherapeutic agents. In particular, combination therapy with palictaxel and modified derivatives (see, e.g., EP0600517) is contemplated. The anti-Lng105 antibody will be administered with a therapeutically effective dose of the chemotherapeutic agent. In another embodiment, the anti-Lng105 antibody is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

In one particular embodiment, an immunoconjugate comprising the anti-Lng105 antibody conjugated with a cytotoxic agent is administered to the patient. Preferably, the immunoconjugate bound to the Lng105 protein is internalized by the cell, resulting in increased therapeutic efficacy of the immunoconjugate in killing the cancer cell to which it binds. In a preferred embodiment, the cytotoxic agent targets or interferes with the nucleic acid in the cancer cell. Examples of such cytotoxic agents are described above and include maytansinoids, calicheamicins, ribonucleases and DNA endonucleases.

The anti-Lng105 antibodies or immunoconjugate are administered to a human patient, in accord with known methods, such as intravenous administration, e.g., as a bolus or by continuous infusion over a period of time, by intramuscular, intraperitoneal, intracerebrospinal, subcutaneous, intra-articular, intrasynovial, intrathecal, oral, topical, or inhalation routes. Intravenous or subcutaneous administration of the antibody is preferred. Other therapeutic regimens may be combined with the administration of the anti-Lng105 antibody.

The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-Lng105 antibody or antibodies, with administration of an antibody directed against another tumor antigen associated with the particular cancer.

In another embodiment, the antibody therapeutic treatment method of the present invention involves the combined administration of an anti-Lng105 antibody (or antibodies) and one or more chemotherapeutic agents or growth inhibitory agents, including co-administration of cocktails of different chemotherapeutic agents. Chemotherapeutic agents include estramustine phosphate, prednimustine, cisplatin, 5-fluorouracil, melphalan, cyclophosphamide, hydroxyurea and hydroxynreataxanes (such as paclitaxel and doxetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M.C. Perry, Williams & Wilkins, Baltimore, Md. (1992).

The antibody may be combined with an anti-hormonal compound; e.g., an anti-estrogen compound such as tamoxifen; an anti-progesterone such as onapristone (see, EP 616 812); or an anti-androgen such as flutamide, in dosages known for such molecules. Where the cancer to be treated is androgen independent cancer, the patient may previously have been subjected to anti-androgen therapy and, after the cancer becomes androgen independent, the anti-Lng105 antibody (and optionally other agents as described herein) may be administered to the patient.

Sometimes, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy, before, simultaneously with, or post antibody therapy. Suitable dosages for any of the above co-administered agents are those presently used and may be lowered due to the combined action (synergy) of the agent and anti-Lng105 antibody.

For the prevention or treatment of disease, the dosage and mode of administration will be chosen by the physician according to known criteria. The appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Preferably, the antibody is administered by intravenous infusion or by subcutaneous injections. Depending on the type and severity of the disease, about 1 pg/kg to about 50 mg/kg body weight (e.g. about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-Lng105 antibody. However, other dosage regimens may be useful. A typical daily dosage might range from about 1 pg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

Aside from administration of the antibody protein to the patient, the present application contemplates administration of the antibody by gene therapy. Such administration of nucleic acid encoding the antibody is encompassed by the expression “administering a therapeutically effective amount of an antibody”. See, for example, WO 96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et at., Science 256:808-813 (1992). See also WO 93/25673 and the references cited therein.

Articles of Manufacture and Kits

Another embodiment of the invention is an article of manufacture containing materials useful for the treatment of anti-Lng105 expressing cancer, in particular ovarian, pancreatic, lung or breast cancer. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the cancer condition and may have a sterile access port (for example the container may be an intravenous solution bag or a vial having a stopper pierceable by a hypodermic injection needle). At least one active agent in the composition is an anti-Lng105 antibody of the invention. The label or package insert indicates that the composition is used for treating ovarian, pancreatic, lung or breast cancer, or more specifically ovarian serous adenocarcinoma or breast infiltrating ductal carcinoma cancer. The label or package insert will further comprise instructions for administering the antibody composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes , e.g., for Lng105 cell killing assays, for purification or immunoprecipitation of Lng105 from cells. For isolation and purification of Lng105, the kit can contain an anti-Lng105 antibody coupled to beads (e.g., sepharose beads). Kits can be provided which contain the antibodies for detection and quantitation of Lng105 in vitro, e.g. in an ELISA or a Western blot. As with the article of manufacture, the kit comprises a container and a label or package insert on or associated with the container. The container holds a composition comprising at least one anti-Lng105 antibody of the invention. Additional containers may be included that contain, e.g., diluents and buffers, control antibodies. The label or package insert may provide a description of the composition as well as instructions for the intended in vitro or diagnostic use.

### EXAMPLES

### Example 1

### Production and Selection of Anti-Lng105 Monoclonal Antibodies

The following MAb/hybridomas of the present invention are described below: Lng105.D3, Lng105.D4, Lng105.D6, Lng105.D7, Lng105.D11, Lng105.D12, Lng105.D13, Lng105.D14, Lng105.D16, Lng105.D17, Lng105.D18, Lng105.D19, Lng105.D20, Lng105.D22, Lng105.D27, Lng105.D28, Lng105.D31, Lng105.D32, Lng105.D36, Lng105.D37, Lng105.D39, Lng105.D40, Lng105.D42, Lng105.D44, Lng105.D45, Lng105.D47, Lng105.D48, Lng105.D49, Lng105.D51, Lng105.D53, Lng105.D54, Lng105.D63, Lng105.D65, Lng105.D71, Lng105.D73, Lng105.D74, Lng105.D79, Lng105.J3, Lng105.J6, Lng105.J10, Lng105.J13, Lng105.J15, Lng105.J16, Lng105.J21, Lng105.J23, Lng105.J25, Lng105.J31, Lng105.J32, Lng105.J41, Lng105.J43, Lng105.J50, Lng105.J54, Lng105.J57, Lng105.J64, Lng105.J71, Lng105.J85, Lng105.J86, Lng105.J87, Lng105.J88, Lng105.J91, Lng105.J93, Lng105.J96, Lng105.J99, Lng105.J100, Lng105.J101, Lng105.J104, Lng105.J105, Lng105.J106, Lng105.J109, Lng105.J111, Lng105.J112, Lng105.J114, Lng105.J115, Lng105.J237, Lng105.J238, Lng105.J250, Lng105.J255, Lng105.J263, Lng105.J264, Lng105.J265, Lng105.J276 and Lng105.J281.

If the MAb has been cloned, it will get the nomenclature “X.1,” e.g., the first clone of A7 will be referred to as A7.1, the second clone of A7 will be referred to as A7.2, etc. For the purposes of this invention, a reference to A7 will include all clones, e.g., A7.1, A7.2, etc. An alternative nomenclature format without the “period” (.) punctuation between “Lng105” and the hybridoma may be employed and denotes the same MAb/hybridoma as one with the “period” (.) punctuation.

Immunogens and Antigens (Recombinant Proteins & Transfected Cells)

Lng105 Insect-Expressed Sequence & Protein Production

A PCR fragment of cDNA encoding Ile27 to Gly420 of Lng105 was introduced into an expression vector via recombination. The construct was cloned in-frame to a stanniocalcin 1 secretion signal peptide (Metl to Thr18) at the N-terminal end and a six-histidine tag, located at the C-terminal end with a Gly from the polylinker inserted after Thr18. The resulting Lng105 construct was expressed as six-histidine tagged protein of 419 amino acids. The recombinant plasmid was used to transform competent cells for generation of the infection vector by transposition. The Lng105 recombinant vector was expressed in insect cell lines.

Lng105 Construct Sequence (SEQ ID NO:1):
MLQNSAVLLVLVISASATGIRIPLHRVQPGRRILNLLRGWREPAELPKLG
APSPGDKPIFVPLSNYRDVQYFGEIGLGTPPQNFTVAFDTGSSNLWVPSR
RCHFFSVPCWLHHRFDPKASSSFQANGTKFAIQYGTGRVDGILSEDKLTI
GGIKGASVIFGEALWEPSLVFAFAHFDGILGLGFPILSVEGVRPPMDVLV
EQGLLDKPVFSFYLNRDPEEPDGGELVLGGSDPAHYIPPLTFVPVTVPAY
WQIRMERVKVGPGLTLCAKGCAAILDTGTSLITGPTEEIRALHAAIGGIP
LLTGEYIILCSEIPKLPAVSFLLGGVWFNLTAHDYVIQTTRNGVRLCLSG
FQALDVPPPAGPFWILGDVFLGTYVAVFDRGDMKSSARVGLARARTRGAD
LGWGETAQAQFPGHHHHHH

Cells expressing Lng105 were lysed by sonication in a buffer containing 50 mM Tris/HCl, pH 7.4, 250 mM NaCl and 10 mM CHAPS. The extracts were centrifuged at about 30,600 g and the recovered pellets were dissolved in a strong chaotropic buffer containing 6 M guanidium/HCl, 10 mM imidazole, 5 mM β-mercaptoethanol and 0.1 M Na2HPO3/NaH2PO3, pH 8.1. The suspended samples were stirred overnight at room temperature and then clarified by centrifugation and filtration. The supernatants were loaded onto a Ni-NTA column, equilibrated with a buffer containing 6 M guanidium/HCl, 10 mM imidazole, 5 mM β-mercaptoethanol and 0.1 M Na2HPO3/NaH2PO3, pH 8.0. The columns were then washed with the same buffers with increasing concentration of imidazole. The most stringent wash contained 60 mM imidazole. Following the elution, proteins were precipitated by dialysis against PBS, pH 7.2, and used as a homogenized suspension.

Lng105 Mammalian-Expressed Sequence & Protein Production

A PCR fragment of cDNA encoding Thr25 to Gly420 of Lng105 was introduced into an expression vector via recombination. The construct was cloned in-frame to a stanniocalcin 1 secretion signal peptide (Met1 to Gln23) at the N-terminal end and a six-histidine tag, located at the C-terminal end. Additionally, due to polylinkers in the vector the construct contains 4 residues, SRTL, following the first 18 amino acid STC secretion signal and 11 residues, ASYPYDVPDYA, before the six-histidine tag. The resulting Lng105 construct was expressed as six-histidine tagged protein of 438 amino acids. The recombinant plasmid was used to transfect HEK 293F cells for producing proteins.

Lng105 Construct Sequence (SEQ ID NO:2):
MLQNSAVLLVLVISASATHEAEQSRTLIRIPLHRVQPGRRILNLLRGWRE
PAELPKLGAPSPGDKPIFVPLSNYRDVQYFGEIGLGTPPQNFTVAFDTGS
SNLWVPSRRCHFFSVPCWLHHRFDPKASSSFQANGTKFAIQYGTGRVDGI
LSEDKLTIGGIKGASVIFGEALWEPSLVFAFAHFDGILGLGFPILSVEGV
RPPMDVLVEQGLLDKPVFSFYLNRDPEEPDGGELVLGGSDPAHYIPPLTF
VPVTVPAYWQIRMERVKVGPGLTLCAKGCAAILDTGTSLITGPTEEIRAL
HAAIGGIPLLTGEYIILCSEIPKLPAVSFLLGGVWFNLTAHDYVIQTTRN
GVRLCLSGFQALDVPPPAGPFWILGDVFLGTYVAVFDRGDMKSSARVGLA
RARTRGADLGWGETAQAQFPGASYPYDVPDYAHHHHHH

Recombinant mammalian Lng105 was harvested from both the media and cells of a suspension culture. Concentrated culture media were exchanged into PBS, pH 7.9, by diafiltration and cells were lysed in 100 mM Na2HPO3/NaH2PO3, pH 8.0, containing 0.4 M NaCl, 10% glycerol, 1% Triton X-100, and 10 mM imidazole. Following the buffer exchange or lysis, the sample was centrifuged and the supernatant was filtered through a 10 nm cut off filter. The filtered sample was then incubated with MagneHis beads at 4° C. for 2 hr. The beads were washed with 50 mM sodium phosphate buffer, pH 7.9, containing 0.5 M NaCl and different concentrations of imidazole (0, 10 and 20 mM respectively) three times. The protein was then eluted in the same buffer containing 0.5 M imidazole three times. Impure fractions were pooled, dialyzed in the lysing buffer and repeated the same purification procedure again. Alternatively, the extracts were loaded onto a His-Select-Co column and the intended Lng105 was bound on the column efficiently. The column was washed with 50 mM sodium phosphate buffer, pH 7.8, containing 0.5 M NaCl and 20 mM imidazole. Lng105 was eluted by step washing with 50, 100, 500 and 1000 mM imidazole respectively in the same buffer. Purified Lng105 was pooled, concentrated and dialyzed into PBS, pH 7.4, containing 120 mM imidazole and 35% glycerol. The materials were stored at −30 to −20° C.

Immunizations

Groups of 8 BALB/c mice were immunized intradermally in both rear footpads for generation of the D and J series antibodies. For the D series, mice were immunized with denatured, insect-derived Lng105 recombinant protein (SEQ ID NO: 1). The mice from the J series were immunized with native, mammalian-derived Lng105 recombinant protein (SEQ ID NO: 2). The first injections consisted of 5 ug of insect-derived Lng105 protein for the D series and 10 ug mammalian-derived Lng105 protein for the J series per mouse mixed with varying amounts of Titermax gold adjuvant (Sigma, Saint Louis, Miss.) and DPBS, which was based on the protein concentration. All injections were comprised of a total of 25 uL per footpad. After the first injection, mice were immunized twice weekly for 5 weeks. For the 2^{nd} through 9^{th} injections, mice were immunized using 10 ug of protein and a mixture of Adju-phos adjuvant (Accurate Chemical & Scientific Corp., Westbury, N.Y.) and DPBS. The final immunization consisted of 10 ug of protein in DPBS alone.

Hybridoma Fusions

Mice were sacrificed at the completion of the immunization protocol and draining lymph node (popliteal) tissue was collected by sterile dissection. Lymph node cells were dispersed using a Tenbroeck tissue grinder (Wheaton #357426, VWR, Brisbane, Calif.) followed by pressing through a sterile 40 uM sieve (VWR) into DMEM and removing T-cells via anti-CD90 (Thyl.2) coated magnetic beads (Miltenyl Biotech, Baraisch-Gladbach, Germany).

These primary B-cell enriched lymph node cells were then immortalized by electro-cell fusion (BTX, San Diego, Calif.) with the continuous myeloma cell line P3×63Ag8.653 (Kearney, J. F. et al., J. Immunology 123: 1548-1550, 1979). Successfully fused cells were selected by culturing in standard Hypoxanthine, Azaserine (HA) (Sigma, St. Louis, Mo.) containing selection medium (DMEM/15% FBS/0.5 ng/mL rIL-6 (Sigma)/10% P388D, (ATCC, Manassas, Va.) conditioned medium/OPI (Sigma)). These fusion cultures were distributed at approximately 2 million cells per plate into 96-well culture plates (Costar Cat. #3585, VWR).

Luminex Pairing Assay Screening & Evaluation

Using a multiplexed six-layer sandwich approach, monoclonal antibody pairs were screened for use in a sandwich immunoassay format

Rat anti-mouse IgG MAb cocktail (RDI-LOMG-COC2, Research Diagnostics Inc, Flanders N.J., USA) was carbodiimide coupled to Luminex MAP® Multi-Analyte COOH Microspheres (beads) (L100-Cxxx-01, Luminex Corp. Austin Tex., USA) that are internally labeled with a combination of dyes that allow for the multiplexing of up to 1 00 different bead classifications in a single assay. Antigen-specific test capture MAb was bound by the anti-mouse IgG cocktail antibody (one test antibody per bead classification dye) after blocking non-specific binding with Tris-Buffered-Saline with 0.5% BSA (TBST/BSA). The beads for a series of test capture MAbs were then washed and pooled together. The pooled beads were incubated with specific antigen, washed and aliquoted for incubation with a series of antigen-specific test label MAbs. The label MAbs were then indirectly bound to Phycoerythrin (PE) by incubating first with biotinylated rat anti-mouse IgG MAb cocktail(RDI-LOMG-COC2-bt, Research Diagnostics Inc) followed by streptavidin-R-phycoerythrin.conjugate (SAPE) (S866, Molecular Probes, Eugene Oreg.,USA).

Tris-buffered saline with 5% BSA (TBST/BSA) was used as block buffer and diluent for all incubation steps. The test capture and label MAb reagents were either diluted protein A purified MAbs or unpurified hybridoma tissue culture supernatant samples. The specific antigen prep was either purified Lng105 protein or concentrated culture supernatant from Lng105 transfected 293 cells.

The bead aliquots were read with a Luminex 100 reader. The microspheres from a single sample (containing multiple test capture MAbs and single test label MAb) were introduced into a laminar fluid flow where each bead was sequentially excited by two lasers. One laser excited the PE molecular tag and the second laser excited the microsphere dye allowing for the identification of the particular bead set. Median PE fluorescence intensity associated with each bead set was reported for each sample. High signal intensity is indicative of a more favorable capture MAb and label MAb pair for use in a sandwich immunoassay with the specific antigen. The data were exported to Microsoft Excel and the MAbs were grouped according to similar pairing data profiles.

This evaluation separated the Lng105 D and J series antibodies into groups based upon similar pairing profiles. MAb/hybridomas from each group were selected for sub-cloning, MAb production and purification based on off-rate kinetics as determined by BiaCore evaluation. The purified MAbs selected by off-rate kinetics were then biotinylated and evaluated in a checkerboard ELISA blocking assay to confirm the pairing profiles.

ELISA Screening & Selection of Antibody Producing Hybridomas

Hybridoma cell lines were selected for production of Lng105 specific antibody by enzyme linked solid phase immunoassay (ELISA). Lng105 proteins were nonspecifically adsorbed to wells of 96 well polystyrene EIA plates (VWR). One hundred uL volumes of Lng105 antigen at approximately 1 ug/mL in (DPBS) were incubated overnight at 4° C. in wells of 96 well polystyrene EIA plates. Plates were washed twice with Tris buffered saline with 0.05% Tween 20, pH 7.4 (TBST). The plate wells were then emptied and nonspecific binding capacity was blocked by completely filling the assay wells with TBST/0.5% bovine serun albumin (TBST/BSA) and incubating for 60 minutes at room temperature (RT). The plate wells were emptied and 100 uL of hybridoma culture medium samples were diluted 1:1 with TBST/BSA was added to the wells and incubated for 1 hour at RT. The wells were then washed 3 times with (TBST). One hundred uL of alkaline phosphatase conjugated goat anti-mouse IgG (Fc) (Pierce Chemical Co., Rockford, Ill.), diluted 1:5000 in TBST/BSA, was then added to each well and incubated for 1 hour at RT. The wells were then washed 3 times with TBST. One hundred uL of alkaline phosphatase substrate para-nitrophenylphosphate (pNPP) (Sigma) at 1 mg/mL in 1 M Diethanolamine buffer pH 8.9 (Pierce, Rockford, Ill.) was then added to each well and incubated for 25 min. at RT. Bound alkaline phosphatase activity was indicated by the development of a visible yellow color. The enzymatic reaction was quantified by measuring the solution's absorbance at 405 nm wavelength. Cultures producing the highest absorbance values were chosen for expansion and farther evaluation.

Results for the D Series

There were 79 hits selected from the primary screen, which was performed using the Lng105 insect-derived protein. The secondary screen included reconfirmation of these positives in addition to a negative screen using pepsin. Of these 79 hits, 37 were frozen down: Lng105.D3, Lng105.D4, Lng105.D6, Lng105.D7, Lng105.D11, Lng105.D12, Lng105.D13, Lng105.D14, Lng105.D16, Lng105.D17, Lng105.D18, Lng105.D19, Lng105.D20, Lng105.D22, Lng105.D27, Lng105.D28, Lng105.D31, Lng105.D32, Lng105.D36, Lng105.D37, Lng105.D39, Lng105.D40, Lng105.D42, Lng105.D44, Lng105.D45, Lng105.D47, Lng105.D48, Lng105.D49, Lng105.D51, Lng105.D53, Lng105.D54, Lng105.D63, Lng105.D65, Lng105.D71, Lng105.D73, Lng105.D74 and Lng105.D79

Of these, 13 antibodies (Lng105.D4, Lng105.D6, Lng105.D7, Lng105.D12, Lng105.D16, Lng105.D18, Lng105.D20, Lng105.D22, Lng105.D28, Lng105.D36, Lng105.D37, Lng105.D44, and Lng105.D63) were selected for further study based on elevated binding to Lng105 and decreased binding to pepsin.

Results for the J Series

There were 71 positive hits in the primary screen, 10 of which were specific to the pro form of Lng105, 1 of which recognized only the mature form of Lng105 and the rest recognized a common region in the Lng105 protein. Ten hybridomas were frozen and subcloned. A second and third set of antibodies were generated using a pool of confirmed hits from the J series. Two vials of the pools were thawed, plated by the Coulter Elite Flow Cytometer at 1 cell/well and tested for reactivity against Lng105. From the first vial, there were 38 positives by ELISA and the second vial yielded 40 positives. The following hybridomas and clones were selected for further testing: Lng105.J3, Lng105.J6, Lng105.J10, Lng105.J13, Lng105.J15, Lng105.J16, Lng105.J21, Lng105.J23, Lng105.J25, Lng105.J31, Lng105.J32, Lng105.J41, Lng105.J43, Lng105.J50, Lng105.J54, Lng105.J57, Lng105.J64, Lng105.J71, Lng105.J85, Lng105.J86, Lng105.J87, Lng105.J88, Lng105.J91, Lng105.J93, Lng105.J96, Lng105.J99, Lng105.J100, Lng105.J101, Lng105.J104, Lng105.J105, Lng105.J106, Lng105.J109, Lng105.J111, Lng105.J112, Lng105.J114, Lng105.J115, Lng105.J237, Lng105.J238, Lng105.J250, Lng105.J255, Lng105.J263, Lng105.J264, Lng105.J265, Lng105.J276 and Lng105.J281.

Both D and J series positive hits were subjected to BiaCore and Luminex analyses to narrow the pool to be tested for use in a sandwich immunoassay format.

Cloning of Selected Hybridomas

Monoclonal cultures, consisting of the genetically uniform progeny from single cells, were established after the screening procedure above by cell sorting of single viable cells into wells of two 96 well plates (VWR), using flow cytometry (Coulter Elite, Beckman Coulter, Miami, Fla.). The resulting murine B-cell hybridoma cultures were expanded using standard tissue culture techniques. Selected hybridomas were cryopreserved in fetal bovine serum (FBS) with 10% DMSO and stored in Liquid Nitrogen at −196° C. to assure maintenance of viable clone cultures.

Lng105 Hybridoma Supernatant Off-Ranking Analyses

Binding off-ranking values were calculated from surface plasmon resonance measurements using a BIACORE 3000 instrument (BiaCore, Piscataway, N.J.). A RAM-Fc surface was used to capture each antibody supernatant, followed by an injection of the Lng105 mature form antigen (lot# lot070103) over the captured antibody.

Flow cell 1 of a CM5 sensor chip (BiaCore) was used as a blank surface for reference subtractions, and was activated and then inactivated with ethanolamine per standard BiaCore protocols. Flow cell 2 was used to immobilize RAM Fc using an injection time of 12 minutes and a flow of 5 ul/min. The RAM-Fc (BiaCore) was diluted to 35 ug/mL in 10 mM acetate as suggested. Standard amine coupling (BiaCore) was used to immobilize 10349 RU. Lng105 D and J supernatants were diluted 1:2 in HBS-EP running buffer (BiaCore) and passed over flow cells 1 and 2. Antibodies were captured at 5 ul/min flow rate, 3 minute injection, and Lng105 m was injected at 5 ug/mL for 2 minutes. The dissociation time was 3 minutes. The regeneration of the chip surface, or removal of captured hybridoma supernatants binding to the antigen between cycles, was performed by injecting 10 mM glycine pH 1.75 for 30 seconds at 100 uL/minute.

The above procedure was performed by using the BiaCore's surface preparation and binding wizard included in the BiaCore control software. The results presented below were automatically fitted using the separate ka/kd function included in the BiaCore analysis software, assuming a 1:1 Langmuir binding model.

| Anti Lng105 clone | kd values |
|---|---|
| Lng105.D18.2 | 1.43e−4 |
| Lng105.D28.5.1 | 1.83e−4 |
| Lng105.J86.1 | 3.08e−5 |
| Lng105.J91 | 1.38e−4 |
| Lng105.J99 | 1.23e−4 |
| Lng105.J101 | 1.64e−5 |
| Lng105.J104 | 9.15e−5 |
| Lng105.J109.1 | 3.81e−5 |
| Lng105.J111 | 4.41e−5 |

The isotypes of the D and J series MAbs were determined using commercially available mouse monoclonal antibody isotyping immunoassay test kits (IsoStrip, Roche Diagnostic Corp., Indianapolis, Ind.). Results of the isotyping are listed in the table below.

Lng105 MAb Isotypes

| MAb | Isotype |
|---|---|
| Lng105.D4.1 | IgG₁ kappa |
| Lng105.D18.2 | IgG₁ kappa |
| Lng105.D28.5.1 | IgG₁ kappa |
| Lng105.J86.1 | IgG₁ kappa |
| Lng105.J109.1 | IgG₁ kappa |

Lng105-D and -J Series MAb Checkerboard ELISA

High binding polystyrene plates (Coming Life Sciences) were coated overnight at 4° C. with 0.8 μg/well of anti-Lng105 MAb. The coating solution was aspirated off and free binding sites were blocked with 300 μl/well Superblock-TBS (Pierce Biotechnology, Illinois) plus 100% Calf serum for lhour at room temperature (RT). After washing 4 times with washing buffer (TBS+0.1% Tween20), 100 μl of Assay Buffer (TBS, 1% BSA, 1% Mouse Serum, 1% Calf Serum, 0.1% Tween20) was added to each well and then 25 μl of antigen was added for 60 minutes incubation. For each sandwich ELISA, standards of 1000, 200, 50, 20, 10, 0 pg/ml Lng105 were run in parallel with the test samples. Standards and test samples were diluted in TBS with 1% BSA. For the detection, 100 μl of biotinylated mAb (1 μg/ml) were added to each well and incubated for 1 hour at room temperature, while shaking. After washing, add 100 μL of AP-Streptavidin conjugate (Jackson Lab) at 1:2000 dilution in TBS, and incubate plate with shaking at RT for 30 min. After washing the plate, add 100 uL/well of pNPP substrate (Pierce) and incubate at RT in dark for 30 minutes. The reaction was stopped using 100 μl/well 1N NaOH, and the plates were read at 405 nm using a Spectramax 190 plate reader (Molecular Devices).

For the checkerboard ELISA, all possible combination of antibodies, were tested for efficiency as coating or detecting reagents. The pairs D18/D28 gave the best signal/noise ratio and were further evaluated in sandwich ELISA assays to analyze the efficiency of detection of endogenous Lng105 in human serum.

Lng105-D Series Results

The results of the checkerboard ELISA on 12 anti-Lng105 mAbs are shown in the Table below. Each antibody was tested as both a coating and detecting antibody, in all possible combination. The results are shown as specific signal/noise ratio. All pairs were tested in duplicates with 100 ng of recombinant Lng105 protein in buffer, with buffer alone as a blank. The results are shown as specific signal/noise ratio. The MAbs detect several distinct epitopes, based on these pairing data. Several pairs with the highest signal/noise ratio were used to test sensitivity for recombinant protein, reactivity towards native protein in cell lines and some initial serum samples. Capture MAbs are listed on the Y-axis with detecting MAbs on the X-axis in the table below.

Pairing of Lng105 D-series mAb by Sandwich ELISA

| | D4 | D12 | D36 | D7 | D18 | D16 | D44 | D28 | D63 | D20 | D22 | D37 |
|---|---|---|---|---|---|---|---|---|---|---|---|---|
| D4 | 1 | 1 | 1 | 3 | 15 | 13 | 11 | 24 | 2 | 3 | 1 | 1 |
| D12 | 1 | 1 | 1 | 3 | 12 | 12 | 9 | 21 | 3 | 2 | 1 | 1 |
| D36 | 1 | 1 | 1 | 3 | 13 | 11 | 8 | 20 | 5 | 2 | 1 | 1 |
| D7 | 6 | 6 | 7 | 1 | 1 | 27 | 21 | 42 | 29 | 3 | 1 | 2 |
| D18 | 6 | 4 | 7 | 1 | 1 | 23 | 16 | 49 | 13 | 2 | 1 | 2 |
| D16 | 5 | 4 | 6 | 6 | 35 | 2 | 6 | 30 | 5 | 2 | 1 | 2 |
| D44 | 3 | 2 | 3 | 12 | 35 | 8 | 1 | 4 | 1 | 2 | 1 | 1 |
| D28 | 3 | 2 | 3 | 5 | 34 | 7 | 1 | 2 | 1 | 2 | 1 | 1 |
| D63 | 6 | 5 | 7 | 19 | 37 | 23 | 1 | 3 | 1 | 3 | 1 | 1 |
| D20 | 2 | 2 | 2 | 1 | 1 | 3 | 3 | 1 | 1 | 1 | 1 | 1 |
| D22 | 2 | 2 | 2 | 1 | 3 | 4 | 2 | 4 | 1 | 1 | 1 | 1 |
| D37 | 1 | 1 | 1 | 1 | 3 | 2 | 1 | 3 | 1 | 1 | 1 | 1 |

Lng105-D and -J Series Results

The results of the checkerboard ELISA on 12 anti-Lng105 mAbs and 2 negative control mAbs (4B4 and TBS) are shown in the table below. Each antibody was tested as both a coating and detecting antibody, in all possible combination. The results are shown as specific signal/noise ratio. All pairs were tested in duplicates with 100 ng of recombinant Lng105 protein in buffer, with buffer alone as a blank. The results are shown as specific signal/noise ratio. The MAbs detect several distinct epitopes, based on these pairing data. Several pairs with the highest signal/noise ratio were used to test sensitivity for recombinant protein, reactivity towards native protein in cell lines and some initial serum samples. Capture MAbs are listed on the Y-axis with detecting MAbs on the X-axis in the table below.

Pairing of Lng105-D and -J series mAb by Sandwich ELISA

| | J86 | J91 | J99 | J101 | J104 | J109 | J111 | D28 | D4 | D18 | D22 | D63 | 4B4 |
|---|---|---|---|---|---|---|---|---|---|---|---|---|---|
| J86 | 16 | 1 | 1 | 27 | 2 | 35 | 17 | 14 | 3 | 35 | 1 | 16 | 1 |
| J91 | 1 | 1 | 1 | 1 | 1 | 1 | 1 | 1 | 1 | 1 | 1 | 1 | 1 |
| J99 | 2 | 1 | 1 | 1 | 1 | 3 | 2 | 2 | 1 | 3 | 1 | 1 | 1 |
| J101 | 40 | 1 | 10 | 14 | 5 | 32 | 40 | 40 | 4 | 33 | 1 | 40 | 1 |
| J104 | 3 | 1 | 1 | 1 | 1 | 3 | 2 | 2 | 1 | 2 | 1 | 1 | 1 |
| J109 | 39 | 2 | 14 | 32 | 7 | 11 | 39 | 38 | 4 | 9 | 1 | 40 | 1 |
| J111 | 14 | 1 | 1 | 28 | 2 | 40 | 14 | 11 | 3 | 40 | 1 | 17 | 1 |
| D28 | 29 | 1 | 1 | 28 | 2 | 38 | 26 | 15 | 3 | 40 | 1 | 9 | 1 |
| D4 | 11 | 1 | 3 | 6 | 2 | 9 | 9 | 7 | 1 | 7 | 1 | 7 | 1 |
| D18 | 37 | 2 | 12 | 31 | 6 | 13 | 39 | 40 | 4 | 11 | 1 | 40 | 1 |
| D22 | 1 | 1 | 1 | 1 | 1 | 1 | 1 | 1 | 1 | 1 | 1 | 1 | 1 |
| D63 | 22 | 1 | 2 | 33 | 2 | 40 | 21 | 18 | 5 | 40 | 1 | 7 | 1 |
| 4B4 | 1 | 1 | 1 | 1 | 1 | 1 | 1 | 1 | 1 | 1 | 1 | 1 | 1 |
| TBS | 1 | 1 | 1 | 1 | 1 | 1 | 1 | 1 | 1 | 1 | 1 | 1 | 1 |

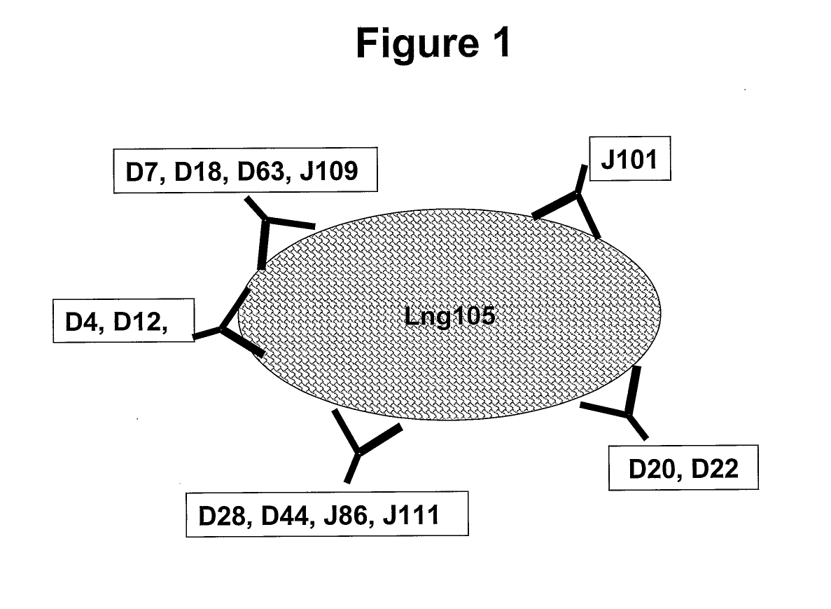

The J-series niAbs (J86, J101, J109, J111) show good binding to recombinant Lng105 in direct ELISA, and J91, J99, J104 bind less strongly. Checkerboard analysis of J-series mAbs with previous D-series mAbs show that J86 and J111 have similar epitope as D28, J109 similar to D18, J101 appear to be unique. The epitope map of the Lng105 MAbs derived from the results in these tables is shown in FIG. 1.
Lng105-D and -J Series MAb Titration Checkerboard ELISA

Serial dilutions of Lng105 were titrated to determine the sensitivity of anti-Lng105 MAbs in an ELISA checkerboard. The results in the table below are shown as the OD read at 405 nm. Capture MAbs are listed on the Y-axis with detecting MAbs on the X-axis in the tables below.

Lng105 concentration: 2000 pg/ml

| | D18 | J109 | D28 | J86 | J111 | J101 |
|---|---|---|---|---|---|---|
| D18 | 1.1 | 1.3 | 10.6 | 16.9 | 13.8 | 8.9 |
| J109 | 1.1 | 1.2 | 13.6 | 22.5 | 19.0 | 12.3 |
| D28 | 11.0 | 15.6 | 1.3 | 1.5 | 1.4 | 3.9 |
| J86 | 13.2 | 19.5 | 1.5 | 1.8 | 1.6 | 5.4 |
| J111 | 12.6 | 18.2 | 1.4 | 1.5 | 1.6 | 5.0 |
| J101 | 12.1 | 13.3 | 7.0 | 12.7 | 10.0 | 1.1 |

Lng105 concentration: 200 pg/ml

| | D18 | J109 | D28 | J86 | J111 | J101 |
|---|---|---|---|---|---|---|
| D18 | 1.1 | 1.1 | 2.1 | 2.8 | 2.5 | 1.8 |
| J109 | 1.0 | 1.0 | 2.4 | 3.5 | 2.8 | 2.2 |
| D28 | 2.1 | 2.7 | 1.0 | 1.1 | 1.0 | 1.3 |
| J86 | 2.4 | 3.2 | 1.1 | 1.1 | 1.0 | 1.4 |
| J111 | 2.3 | 2.9 | 1.1 | 1.0 | 1.1 | 1.3 |
| J101 | 2.2 | 2.2 | 1.6 | 2.3 | 1.7 | 1.0 |

Lng105 concentration: 20 pg/ml

| | D18 | J109 | D28 | J86 | J111 | J101 |
|---|---|---|---|---|---|---|
| D18 | 0.94 | 1.03 | 1.10 | 1.16 | 1.22 | 1.09 |
| J109 | 0.97 | 0.95 | 1.14 | 1.24 | 1.23 | 1.07 |
| D28 | 1.07 | 1.15 | 1.00 | 1.01 | 1.00 | 1.06 |
| J86 | 1.16 | 1.29 | 1.04 | 1.03 | 1.04 | 1.04 |
| J111 | 1.11 | 1.19 | 1.01 | 0.97 | 1.11 | 0.97 |
| J101 | 1.15 | 1.08 | 1.01 | 1.18 | 1.13 | 1.03 |

Anti-Lng105 antibody pairs (capture/detect) D18/D28 and J109/86 are highly specific for Lng105 and demonstrate excellent sensitivity for detection Lng105. These antibody pairs were selected for use in sandwich ELISA detection of Lng105.

### Example 2

### Monoclonal Sandwich ELISA Detection of Lng105

Human Serum Samples

Human cancer and benign serum samples were obtained from IMPATH-BCP, Inc. (Franklin, Mass.) and Diagnostic Support Services, Inc. (West Yarmouth, Mass.). The serum samples from healthy women were obtained from ProMedex, Inc. (Flushing, N.Y.). All samples were aliquoted upon arrival and stored at minus 80° C. until use.

Lng105-D and -J Series MAb ELISA

As described above, for the detection of Lng105 in serum samples, a sensitive detection system based on the use of alkaline phosphatase (AP) and a high sensitivity pNPP substrate (Pierce) was used. The minimal detectable dose (MDD) for Lng105 in this ELISA format is 10 pg/ml. For calculation of median values, samples with values below the MDD were defined as 10 pg/ml Lng105. The minimum detectable dose is defined as two standard abbreviations above the background signal. Most of the serum samples from healthy patients showed low Lng105 concentrations in the sandwich ELISA while sera from ovarian cancer patients have elevated levels of Lng105.

We tested the Lng105 concentration in more than 2700 serum samples from patients with lung, breast, colon, prostate or ovarian cancer or with non-cancerous, benign diseases. An overview of all samples tested is listed in the table below.

Serum Samples Tested by Sandwich ELISA

| Sample Type | No. of Samples |
|---|---|
| Normal | 555 (281-M, 274-F) |
| Breast Cancer | 260 |
| Breast Benign | 180 |
| Colon Cancer | 150 (71-M, 79-F) |
| Colon Benign | 296 (151-M, 145-F) |
| Lung Cancer | 323 (235-M, 93-F) |
| Lung Benign | 250 (130-M, 120-F) |
| Ovarian Cancer | 236 |
| Ovarian Benign | 150 |
| Prostate Cancer | 138 |
| Prostate Benign | 147 |

Lng105 D18/D28 MAb ELISA Results

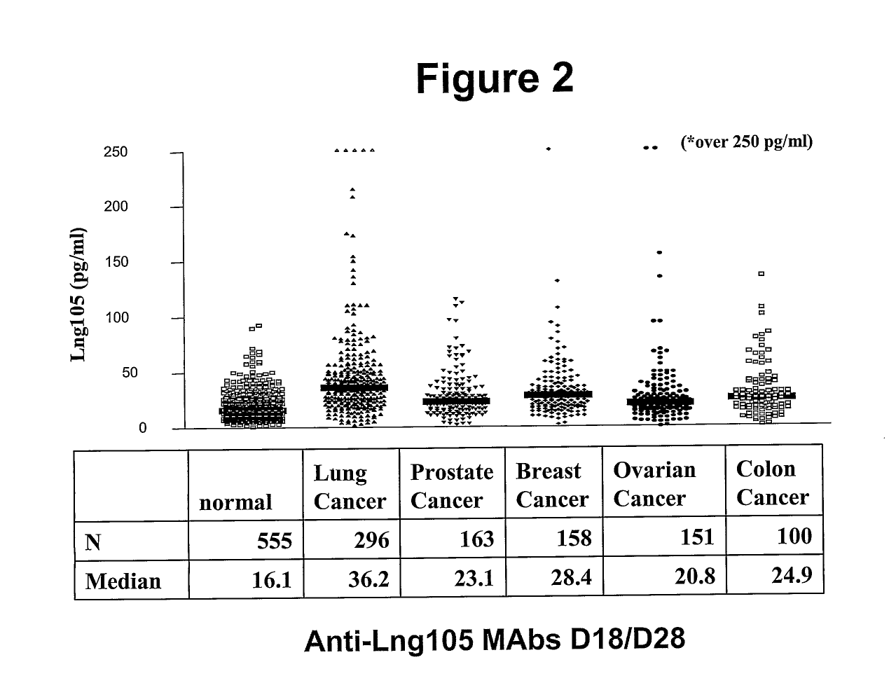

FIG. 2 shows the Lng105 concentration in serum from 555 healthy donors and more than 1200 patients with cancer. Elevated levels of Lng105 are observed in some patients of all cancer types but patients with lung cancer have the highest median Lng105 concentration.

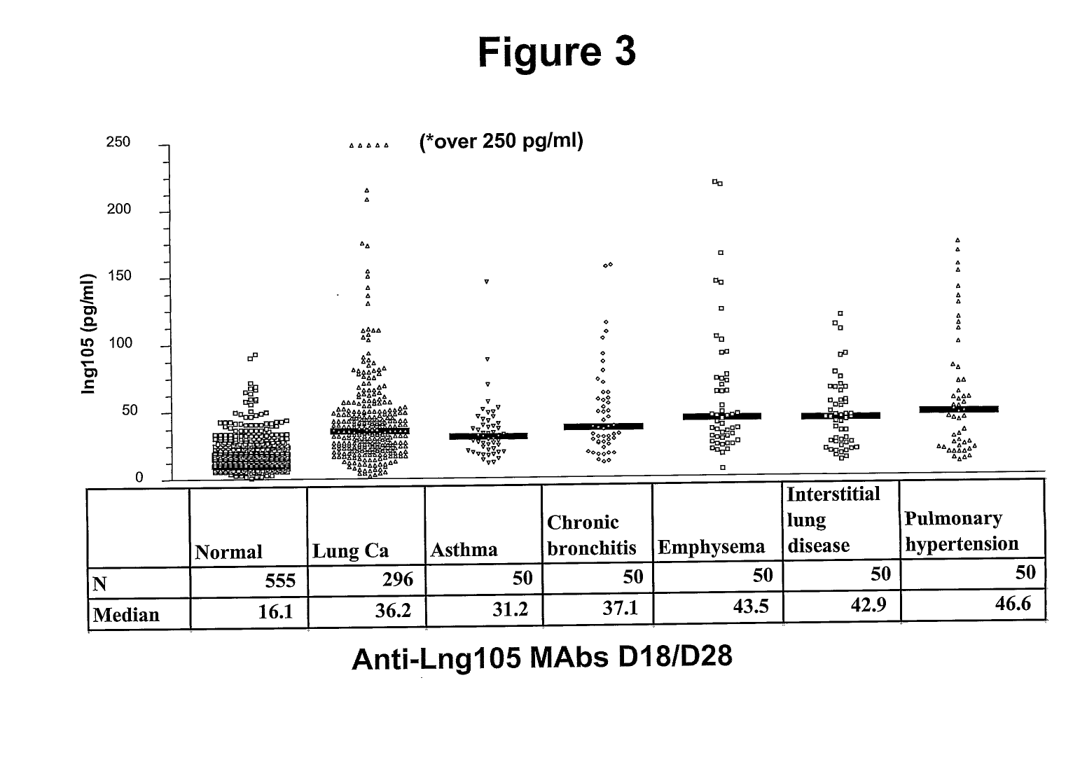

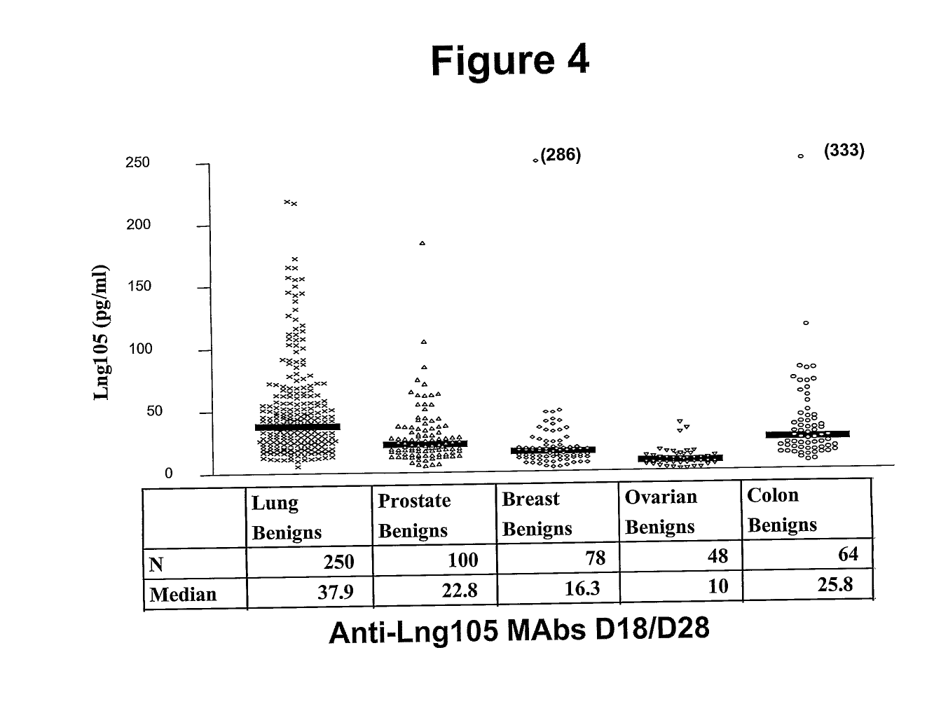

We then tested serum samples from various lung benign diseases patients. The results shown in FIG. 3 indicate that Lng105 was elevated in lung benign conditions. In contrast, Lng105 is moderately elevated in benign diseases from other tissues as shown in FIG. 4.

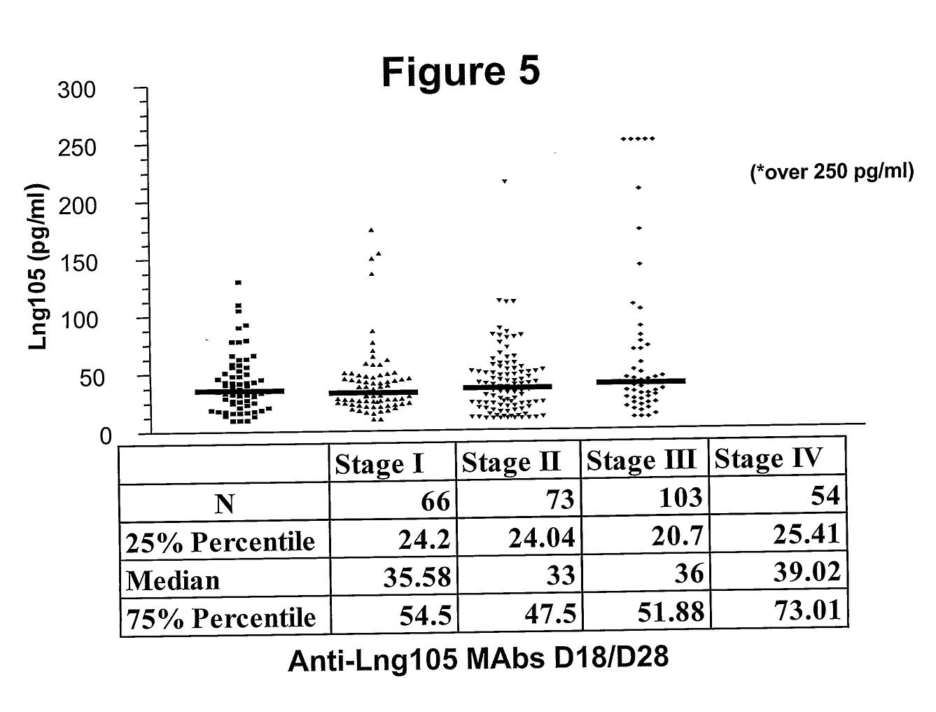

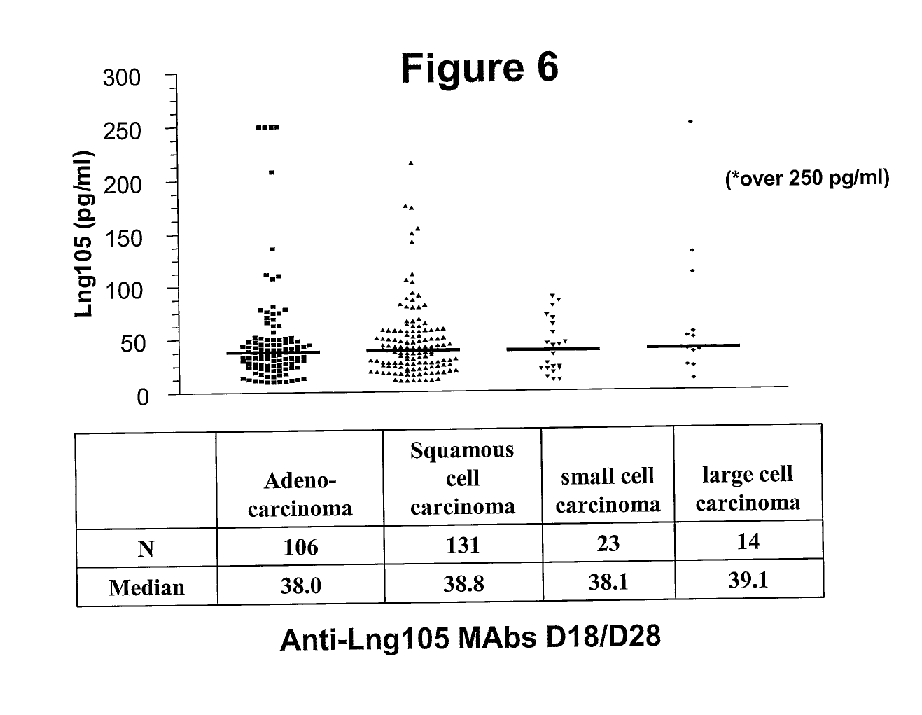

We analyzed Lng105 serum level according to cancer stage and histopathologic type. FIGS. 5 and 6 do not indicate a correlation of Lng105 concentration with a particular lung cancer stage or histopathologic type.

Lng105 J109/86 MAb ELISA Results

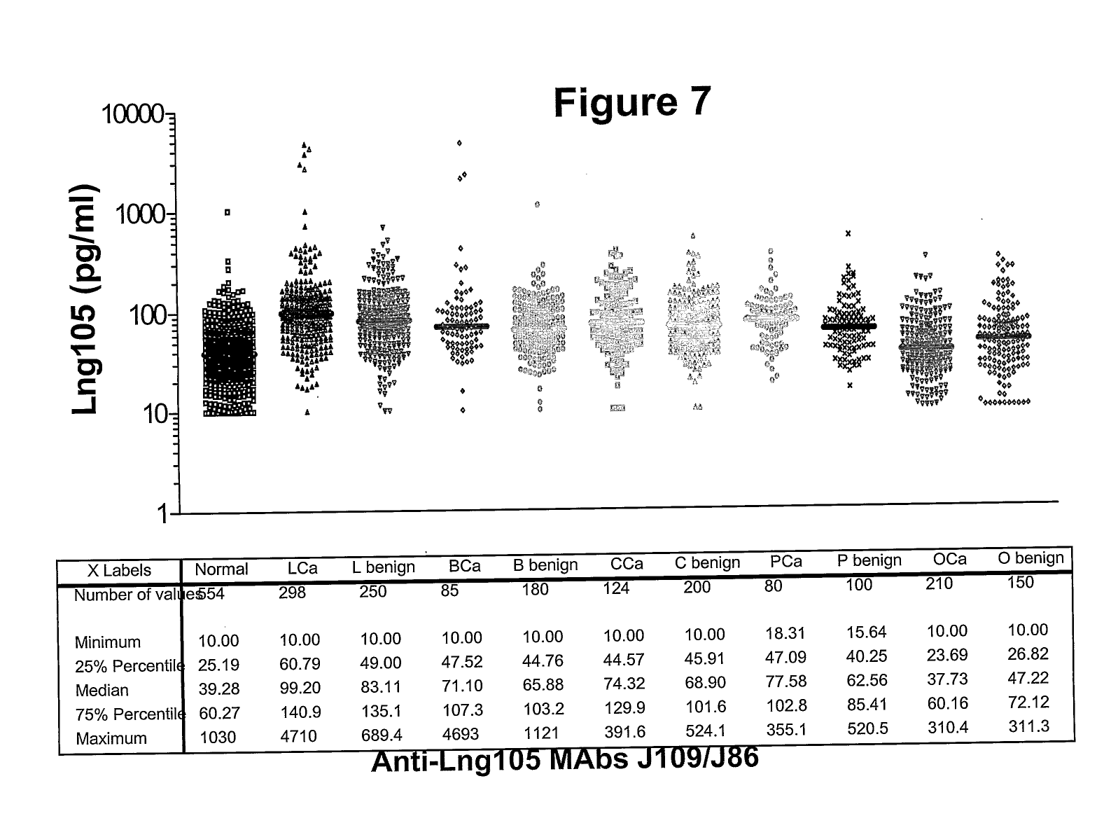

FIG. 7 shows the Lng105 concentration in serum from 554 healthy donors, more than 790 donors with cancer and 880 donors with various benign conditions. In FIG. 7 tissue types are indicated by a single capital letter, L=lung, B=breast, C=colon, P=prostate and O=ovarian. Disease states are indicated as “Ca” for cancer or “benign” for benign conditions. For example, lung cancer samples are indicated as “LCa”.

Elevated levels of Lng105 are observed in some patients of all cancer types but patients with lung cancer have the highest median Lng105 concentration. Lng105 was also elevated in lung benign conditions. In contrast, Lng105 is moderately elevated in benign diseases from other tissues.

In agreement with the results above, the assay using J-series antibodies did not demonstrate a correlation of Lng105 concentration with a particular lung cancer stage or histopathologic type.

The J109/86 assay formatted detected twice as much Lng105 in samples due to the increased sensitivity of the antibody pair.

Correlation between Lng105 D-series and J-series MAb ELISA

Correlation between the values observed between the two assay formats was determined using standard methods. Plotting the values for the samples tested in each assay resulted in a line with the following equation: y=2.2941x+7.386 The R² value was 0.7289 indicating that the J109/J86 MAb Lng105 assay correlated well with the D18/D28 MAb Lng105 assay.

### Example 2

### ROC Analysis of Lng105 Levels in Serum

The ability of a test to discriminate diseased cases from normal cases is evaluated using Receiver Operating Characteristic (ROC) curve analysis (Metz, 1978; Zweig & Campbell, 1993). ROC curves can also be used to compare the diagnostic performance of two or more laboratory or diagnostic tests (Griner et al., 1981).

ROC curve is generated by plotting sensitivity against specificity for each value. From the plot, the area under the curve (AUC) can be determined. The value for the area under the ROC curve (AUC) can be interpreted as follows: an area of 0.84, for example, means that a randomly selected positive result has a test value larger than that for a randomly chosen negative result 84% of the time (Zweig & Campbell, 1993). When the variable under study can not distinguish between the two result groups, i.e. where there is no difference between the two distributions, the area will be equal to 0.5 (the ROC curve will coincide with the diagonal). When there is a perfect separation of the values of the two groups, i.e. there no overlapping of the distributions, the area under the ROC curve equals 1 (the ROC curve will reach the upper left corner of the plot).

The 95% confidence interval for the area can be used to test the hypothesis that the theoretical area is 0.5. If the confidence interval does not include the 0.5 value, then there is evidence that the laboratory test does have an ability to distinguish between the two groups (Hanley & McNeil, 1982; Zweig & Campbell, 1993).

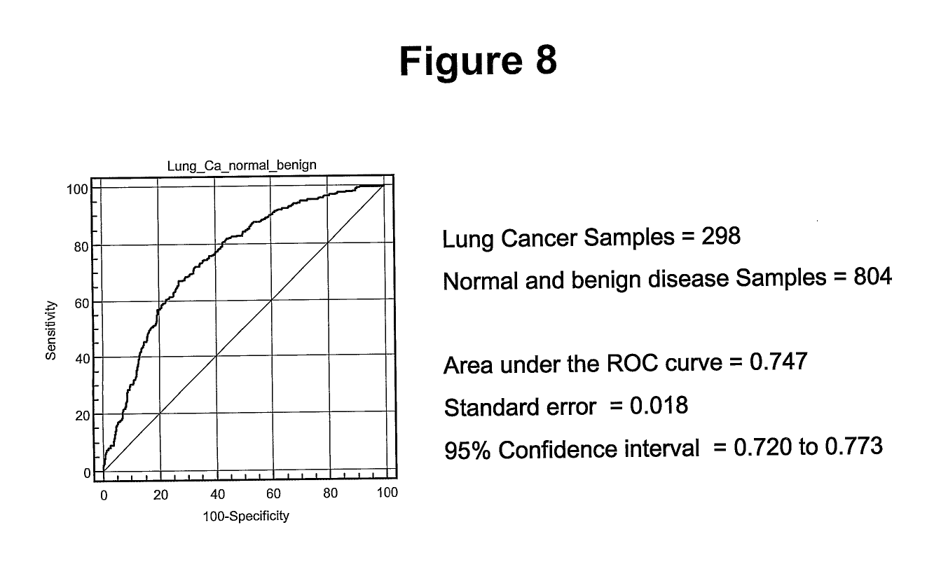

Sensitivity and specificity of the Lng105 assay for detecting cancer was calculated through receiver operating characteristic (ROC) analysis. FIG. 8 shows the ROC analysis of Lng105 comparing lung cancer samples with the normal healthy samples and lung benign diseases demonstrated an area under the curve (AUC) of 0.747.

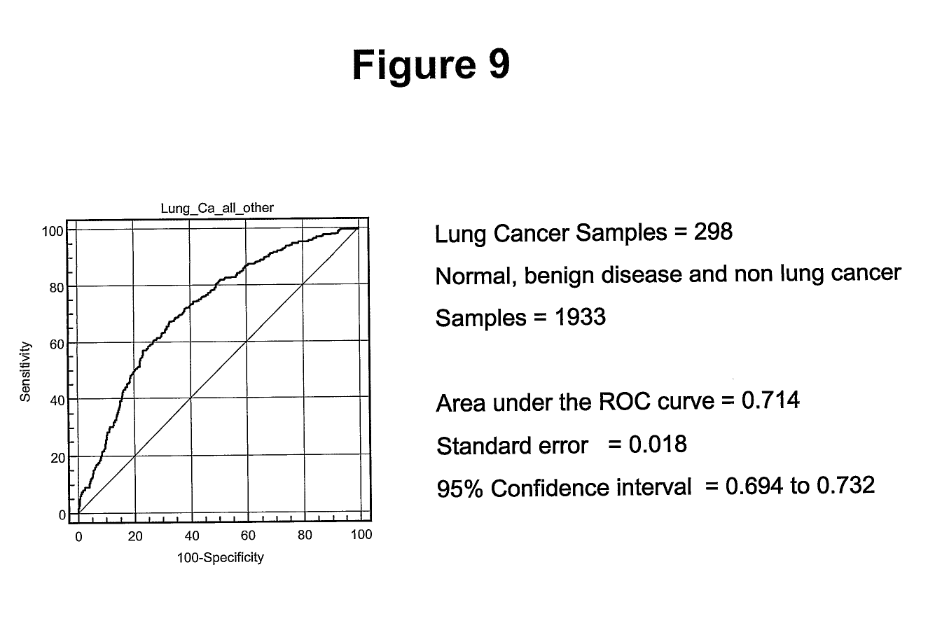

When comparing lung cancer samples with all other non-lung cancer samples (normal, lung benign, other cancer and benign samples), the AUC was 0.714 (FIG. 9).

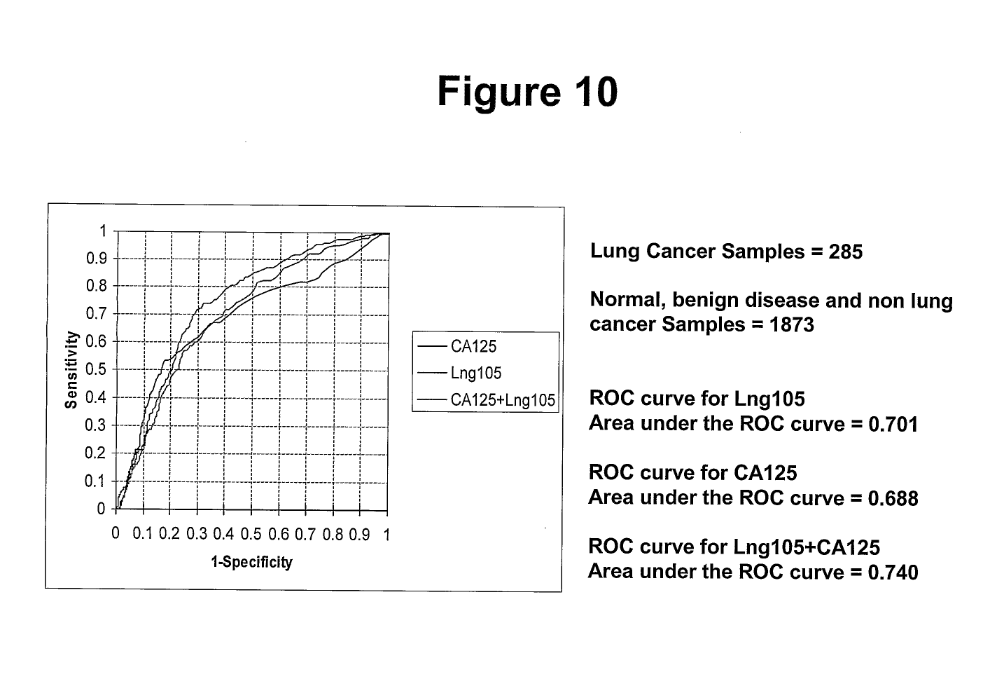

The AUC for Lng105 was compared against know cancer marker, CA125, in lung cancer samples vs. all other non-lung cancer samples (normal, lung benign, other cancer and benign samples). FIG. 10 shows that the AUC for Lng105 is 0.701 and the AUC for CA125 is 0.688 demonstrating the significantly improved sensitivity and specificity of Lng105 over CA125 in detecting lung cancer.

Lng105 was also evaluated in combination with other know cancer makers for the detection of lung cancer samples vs. all other non-lung cancer samples (normal, lung benign, other cancer and benign samples). The table below lists the AUC of each marker alone and in combination with Lng105.

| Lng105 | CEA | Lng105 + CEA | CA125 | Lng105 + CA125 |
|---|---|---|---|---|
| 0.706 | 0.671 | 0.712 | 0.684 | 0.740 |

The AUC of Lng105 alone is higher compared to CEA and CA125, but when evaluated in combination, synergistically the AUC of Lng105+AUC and Lng105+CA125 improve significantly to 0.712 and 0.740, respectively. These results demonstrate that Lng105 alone or in combination with other cancer makers is useful for detecting cancer.

### Example 3

### Immunohistochemical Analysis of Lng105

Immunohistochemical analysis with Lng105 antibodies was performed, specifically, Lng105.D20 (1:4000, f.c.: 0.3 μg/ml) and Lng105.D4 (PTA-6629) (1:2000, f.c.: 0.65 μg/ml). Lng105.D20 and D4 display an identical staining pattern with some exceptions. Evaluation of Lng105.D20 and D4 expression showed strong cytoplasmic and circumferential membranous staining of the cancer cells in 9/9 (100%) cases of pulmonary adenocarcinoma. The proportion of positive cells ranged from about 10% in a few cases but most cases showed staining in 100% of the tumor cells. Intense staining was also detected in reactive type II pneumocytes, histiocytes and alveolar secretions. Bronchial columnar epithelium, lymphocytes and blood vessels were always negative for Lng105 expression. Strong Lng105 expression was detected in alveolar macrophages of all cases of primary pulmonary hypertension and normal lung tissue. Pneumocytes were frequently positive for Lng105 expression. No Lng105 expression was observed in 5/5 cases of pulmonary granuloma. Various cancer types have been evaluated for Lng105: Pancreatic cancer (n=5), colon cancer (n=1), gastric cancer (n=5), renal cell carcinoma (n=4), breast cancer (n=5), and hepatocellular carcinoma (n=10). None of those cancer types showed immunopositivity for Lng105 expression. Adjacent normal convoluted tubules in the kidney showed strong cytoplasmic (granular) staining for Lng105 (D20/D4). Lng105 staining in normal tissues is shown in the table below:

Normal somatic tissues (body panel)

| | expression detected | expression detected |
|---|---|---|
| FDA normal tissue panel | with Lng105.D20 | with Lng105.D4 |
| Abdominal peritoneum | N/E | N/E |
| Adrenal gland | 0/1 (0%) | 0/1 (0%) |
| Amnion | 2/2 (100%) | 2/2 (100%) |
| Blood cells | 0/1 (0%) | 0/1 (0%) |
| Bone marrow | N/E | N/E |
| Breast | 0/5 (0%) | 0/5 (0%) |
| Cerebellum | 0/2 (0%) | 0/2 (0%) |
| Cerebral cortex | 0/2 (0%) | 0/2 (0%) |
| Cervix | 0/5 (0%) | |
| Colon/Cecum | 0/5 (0%) | 0/5 (0%) |
| Duodenum | N/E | N/E |
| Endometrium | N/E | N/E |
| Endothelium | N/E | N/E |
| Esophagus | 2/3 (66.6%) | 0/3 (0%) |
| Eye | 0/1 (0%) | 0/1 (0%) |
| Fallopian tube | N/E | N/E |
| Gallbladder | 0/4 (0%) | 0/4 (0%) |
| Heart | 0/2 (0%) | 0/2 (0%) |
| Ileum | 0/2 (0%) | 0/2 (0%) |
| Kidney | 3/3 (100%) | 3/3 (100%) |
| Liver | 0/3 (0%) | 0/3 (0%) |
| Lung | N/E | N/E |
| Lymph node | 0/5 (0%) | 0/5 (0%) |
| Ovary | N/E | N/E |
| Pancreas | 0/1 (0%) | 0/1 (0%) |
| Parathyroid | N/E | N/E |
| Pituitary gland | 0/2 (0%) | 2/2 (100%) |
| Placenta | 0/2 (0%) | 0/2 (0%) |
| Prostate | N/E | N/E |
| Salivary gland | N/E | N/E |
| Skin | N/E | N/E |
| Spinal cord | 0/5 (0%) | 0/5 (0%) |
| Spleen | 0/3 (0%) | 0/3 (0%) |
| Stomach | 0/3 (0%) | 0/3 (0%) |
| Striated muscle | N/E | N/E |
| Synovial cyst | N/E | N/E |
| Testis | 0/3 (0%) | 0/3 (0%) |
| Thymus | 0/1 (0%) | 0/1 (0%) |
| Thyroid | 0/2 (0%) | 0/2 (0%) |
| Ureter | 0/1 (0%) | 0/1 (0%) |
| Urinary bladder | 0/1 (0%) | 0/1 (0%) |

Lng105 expression was not detected in the great majority of the normal tissues. Specific staining of Lng105 was found in the upper third of the squamous cell layer in the esophagus (D20), the epithelium of the epididymis (D20/D4), some undefined components (possibly endometrial glands) attached to the anion (D20/D4), the scattered parenchymal and glial cells of the pituitary gland (D4), in scattered histiocytes in the lamina propria of the colonic mucosa (D4), and in the majority of the convoluted tubules of the kidney (D20/D4).

### Example 4

### Deposits

Deposit of Cell Lines and DNA The following hybridoma cell lines were deposited with the American Type Culture Collection (ATCC), located at 10801 University Boulevard, Manassas, Va. 20110-2209, U.S.A., and accorded accession numbers. The names of the deposited hybridoma cell lines have been shortened for convenience for reference; these hybridomas correspond to the clones (with their full names) listed in the table below.

ATCC deposits

| Hybridoma | ATCC Accession No. | Deposit Date |
|---|---|---|
| Lng105.D18.2 | PTA-5878 | Mar. 23, 2004 |
| Lng105.D28.5.1 | PTA-5879 | Mar. 23, 2004 |
| Lng105.J86.1 | PTA-6146 | Aug. 4, 2004 |
| Lng105.J109.1 | PTA-6147 | Aug. 4, 2004 |
| Lng105.D4.1 | PTA-6629 | Mar. 11, 2005 |

These deposits were made under the provisions of the Budapest Treaty on the International Recognition of the Deposit of Microorganisms for the Purpose of Patent Procedure and the Regulations there under (Budapest Treaty). This assures maintenance of viable cultures for 30 years from the date of deposit. The organisms will be made available by ATCC under the terms of the Budapest Treaty, and subject to an agreement between diaDexus, Inc. and ATCC, which assures permanent and unrestricted availability of the progeny of the cultures to the public upon issuance o the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 3 7 CFR §1.14 with particular reference to 886 OG 638).

The assignee of the present application has agreed that if the cultures on deposit should die or be lost or destroyed when cultivated under suitable conditions, they will be promptly replaced on notification with a viable specimen of the same culture. Availability of the deposited strains are not to be construed as a license to practice the invention in contravention of the rights granted under the authority of any government in accordance with its patent laws. The making of these deposits is by no means an admission that deposits are required to enable the invention

1. An antibody produced by a hybridoma of American Type Culture Collection accession number PTA-5878, PTA-5879, PTA-6146, PTA-6147 and PTA-6629 or which competes for binding to a same epitope as the epitope bound by the antibody produced by a hybridoma of American Type Culture Collection accession number PTA-5878, PTA-5879, PTA-6146, PTA-6147 and PTA-6629.

2. The antibody of claim 1 which is a monoclonal antibody, an antibody fragment of a chimeric or humanized antibody.

3-4. (canceled)

5. The antibody of claim 1 which is produced by immunization with Lng105 protein.

6. The antibody of claim 1 which binds to native Lng105 protein.

7. (canceled)

8. The antibody of claim 1 further comprising a growth inhibitory agent, an imaging agent or a cytotoxic agent conjugated thereto.

9-10. (canceled)

11. The antibody of claim 8 wherein the cytotoxic agent is selected from the group consisting of toxins, antibiotics, radioactive isotopes and nucleolytic enzymes.

12. (canceled)

13. The antibody of claim 11, wherein the toxin is selected from the group consisting of a ricin, saponin, maytansinoid and calicheamicin.

14. (canceled)

15. The antibody of claim 1 that selectively binds Lng105 in a bodily fluid.

16. The antibody of claim 1 that selectively binds a Lng105-expressing cell.

17. The antibody of claim 1 that inhibits the growth of a Lng105-expressing cell.

18-20. (canceled)

21. The antibody of claim 16, wherein the Lng105-expressing cell is a cancer cell.

22. The antibody of claim 21, wherein the cancer cell is from a cancer comprising lung or breast cancer.

23. (canceled)

24. A cell that produces the antibody of claim 1.

25. (canceled)

26. A method of producing the antibody of claim 1 comprising culturing an appropriate cell and recovering the antibody from the cell culture.

27. A composition comprising the antibody of claim 1, and a carrier.

28. The composition of claim 27, wherein the antibody is conjugated to an imaging agent or a cytotoxic agent.

29. (canceled)

30. The composition of claim 28, wherein the cytotoxic agent is a ricin, saponin, maytansinoid and calicheamicin.

31-33. (canceled)

34. A method of killing an Lng105-expressing cancer cell, comprising contacting the cancer cell with the antibody of claim 1, thereby killing the cancer cell.

35. The method of claim 34, wherein the cancer cell comprises a lung or breast cancer cell.

36. The method of claim 35, wherein the cancer cell is from metastatic lung or breast cancer.

37-38. (canceled)

39. The method of claim 34, wherein the antibody is conjugated to a cytotoxic agent.

40. The method of claim 39, wherein the cytotoxic agent is a toxin selected from the group consisting of maytansinoid, ricin, saporin and calicheamicin or a radioactive isotope.

41-42. (canceled)

43. A method of alleviating a Lng105-expressing cancer in a mammal, comprising administering a therapeutically effective amount of the antibody of claim 1 to the mammal.

44. The method of claim 43, wherein the cancer comprises lung or breast cancer.

45-47. (canceled)

48. The method of claim 43, wherein the antibody is administered in conjunction with at least one chemotherapeutic agent.

49. The method of claim 48 wherein the chemotherapeutic agent is paclitaxel or a derivative thereof.

50. An article of manufacture comprising a container and a composition contained therein, wherein the composition comprises an antibody of claim 1.

51. The article of manufacture of claim 50 further comprising a package insert indicating that the composition can be used to diagnose, image or treat lung or breast cancer.

52. A method for determining if cells in a sample express Lng105 comprising
(a) contacting a sample of cells with the antibody of claim 1 under conditions suitable for specific binding of the antibody to Lng105 and
(b) determining the level of binding of the antibody of claim 1 to cells in the sample, or the level of antibody internalization of the antibody of claim 1 by cells in said sample,
wherein antibody binding of the antibody of claim 1 to cells in the sample or internalization of the antibody of claim 1 by cells in the sample indicate cells in the sample express Lng105.

53-55. (canceled)

56. The method of claim 52 wherein the cancer comprises a lung or breast cancer.

57. (canceled)

58. A method for detecting Lng105 overexpression in a test cell sample, comprising:
(a) combining a test cell sample with the antibody of claim 1 under conditions suitable for specific binding of the antibody of claim 1 to Lng105 expressed by cells in said test sample;
(b) determining the level of binding of the antibody of claim 1 to the cells in the test sample; and
(c) comparing the level of antibody of claim 1 bound to the cells in step (b) to the level of antibody binding of the antibody of claim 1 to cells in a control cell sample,
wherein an increase in the binding of the antibody of claim 1 in the test cell sample as compared to the control is indicative of Lng105 overexpression by cells in the test cell sample.

59-61. (canceled)

62. A method for detecting Lng105 overexpression in a subject in need thereof comprising:
(a) combining a bodily fluid sample of a subject with the antibody of claim 1 under conditions suitable for specific binding of the antibody of claim 1 to Lng105 in said serum sample;
(b) determining the level of Lng105 in the bodily fluid sample; and
(c) comparing the level of Lng105 determined in step (b) to the level of Lng105 in a control;
wherein an increase in the level of Lng105 in the bodily fluid sample from the subject as compared to the control is indicative of Lng105 overexpression in the subject.

63-66. (canceled)

67. The method of claim 62 wherein the method utilizes a plurality of anti-Lng105 antibodies.

68. The method of claim 67 wherein the antibodies are produced by a hybridoma selected from the group comprising PTA-5878, PTA-5879, PTA-6146 and PTA-6147.

69-70. (canceled)

71. A screening method for antibodies that bind to an epitope which is bound by an antibody of claim 1 comprising,:
(a) combining a Lng105-containing sample with a test antibody and an antibody of claim 1 to form a mixture;
(b) determining the level of antibody of claim 1 bound to Lng105 in the mixture; and
(c) comparing the level of antibody of claim 1 bound in the mixture of step (a) to a control mixture;
wherein the level of antibody binding of the antibody of claim 1 to Lng105 in the mixture as compared to the control is indicative of the test antibody's binding to a same epitope that is bound by the antibody of claim 1.

72-76. (canceled)


SEQUENCE LISTING (2 sequences)

SEQ ID NO: 1 - protein, 419 aa, Homo sapien
MLQNSAVLLVLVISASATGIRIPLHRVQPGRRILNLLRGWREPAELPKLGAPSPGDKPIFVPLSNYRDVQYFGEIGLGTPPQNFTVAFDTGSSNLWVPSRRCHFFSVPCWLHHRFDPKASSSFQANGTKFAIQYGTGRVDGILSEDKLTIGGIKGASVIFGEALWEPSLVFAFAHFDGILGLGFPILSVEGVRPPMDVLVEQGLLDKPVFSFYLNRDPEEPDGGELVLGGSDPAHYIPPLTFVPVTVPAYWQIRMERVKVGPGLTLCAKGCAAILDTGTSLITGPTEEIRALHAAIGGIPLLTGEYIILCSEIPKLPAVSFLLGGVWFNLTAHDYVIQTTRNGVRLCLSGFQALDVPPPAGPFWILGDVFLGTYVAVFDRGDMKSSARVGLARARTRGADLGWGETAQAQFPGHHHHHH

SEQ ID NO: 2 - protein, 438 aa, Artificial sequence
  Synthetic
MLQNSAVLLVLVISASATHEAEQSRTLIRIPLHRVQPGRRILNLLRGWREPAELPKLGAPSPGDKPIFVPLSNYRDVQYFGEIGLGTPPQNFTVAFDTGSSNLWVPSRRCHFFSVPCWLHHRFDPKASSSFQANGTKFAIQYGTGRVDGILSEDKLTIGGIKGASVIFGEALWEPSLVFAFAHFDGILGLGFPILSVEGVRPPMDVLVEQGLLDKPVFSFYLNRDPEEPDGGELVLGGSDPAHYIPPLTFVPVTVPAYWQIRMERVKVGPGLTLCAKGCAAILDTGTSLITGPTEEIRALHAAIGGIPLLTGEYIILCSEIPKLPAVSFLLGGVWFNLTAHDYVIQTTRNGVRLCLSGFQALDVPPPAGPFWILGDVFLGTYVAVFDRGDMKSSARVGLARARTRGADLGWGETAQAQFPGASYPYDVPDYAHHHHHH